(19) United States
(12) Patent Application Publication
(10) Pub. No.: US 2023/0042637 A1
(43) Pub. Date: Feb. 9, 2023

(54) Intrasacular Aneurysm Occlusion Device with a Proximal-to-Distal Stack of Shape-Changing Embolic Structures

(71) Applicant: Robert A. Connor, St. Paul, MN (US)

(72) Inventor: Robert A. Connor, St. Paul, MN (US)

(73) Assignee: Aneuclose LLC, St. Paul, MN (US)

(21) Appl. No.: 17/970,510

(22) Filed: Oct. 20, 2022

Related U.S. Application Data
(63) Continuation-in-part of application No. 17/965,502, filed on Oct. 13, 2022.
Application No. 17/965,502 is a continuation-in-part of application No. 17/829,313, filed on May 31, 2022.
Application No. 17/965,502 is a continuation-in-part of application No. 17/476,845, filed on Sep. 16, 2021.
Application No. 17/829,313 is a continuation-in-part of application No. 17/485,390, filed on Sep. 25, 2021, now Pat. No. 11,471,164.
Application No. 17/829,313 is a continuation-in-part of application No. 17/476,845, filed on Sep. 16, 2021.
Application No. 17/829,313 is a continuation-in-part of application No. 17/472,674, filed on Sep. 12, 2021, now abandoned.
Application No. 17/829,313 is a continuation-in-part of application No. 17/467,680, filed on Sep. 7, 2021, now abandoned.
Application No. 17/829,313 is a continuation-in-part of application No. 17/466,497, filed on Sep. 3, 2021, now Pat. No. 11,357,511.
Application No. 17/829,313 is a continuation-in-part of application No. 17/353,652, filed on Jun. 21, 2021, now abandoned.
Application No. 17/829,313 is a continuation-in-part of application No. 17/220,002, filed on Apr. 1, 2021, now Pat. No. 11,464,518.
Application No. 17/829,313 is a continuation-in-part of application No. 17/214,827, filed on Mar. 27, 2021, now Pat. No. 11,484,322.
Application No. 17/829,313 is a continuation-in-part of application No. 17/211,446, filed on Mar. 24, 2021, now abandoned.
Application No. 17/220,002 is a continuation-in-part of application No. 17/214,827, filed on Mar. 27, 2021, now Pat. No. 11,484,322.
Application No. 17/220,002 is a continuation-in-part of application No. 17/211,446, filed on Mar. 24, 2021, now abandoned.
Application No. 17/220,002 is a continuation-in-part of application No. 16/693,267, filed on Nov. 23, 2019, now abandoned.
Application No. 17/220,002 is a continuation-in-part of application No. 16/660,929, filed on Oct. 23, 2019, now Pat. No. 11,471,163.
Application No. 16/693,267 is a continuation-in-part of application No. 16/660,929, filed on Oct. 23, 2019, now Pat. No. 11,471,163.
Application No. 16/693,267 is a continuation-in-part of application No. 16/541,241, filed on Aug. 15, 2019, now abandoned.
Application No. 16/693,267 is a continuation-in-part of application No. 15/865,822, filed on Jan. 9, 2018, now Pat. No. 10,716,573.
Application No. 16/693,267 is a continuation-in-part of application No. 15/861,482, filed on Jan. 3, 2018, now abandoned.
Application No. 16/660,929 is a continuation-in-part of application No. 16/541,241, filed on Aug. 15, 2019, now abandoned.
Application No. 16/660,929 is a continuation-in-part of application No. 15/865,822, filed on Jan. 9, 2018, now Pat. No. 10,716,573.
Application No. 16/660,929 is a continuation-in-part of application No. 15/861,482, filed on Jan. 3, 2018, now abandoned.
Application No. 16/660,929 is a continuation-in-part of application No. 15/865,822, filed on Jan. 9, 2018, now Pat. No. 10,716,573.
Application No. 16/660,929 is a continuation-in-part of application No. 15/081,909, filed on Mar. 27, 2016, now abandoned.
Application No. 16/660,929 is a continuation-in-part of application No. 14/526,600, filed on Oct. 29, 2014, now abandoned.
Application No. 15/861,482 is a continuation-in-part of application No. 15/080,915, filed on Mar. 25, 2016, now Pat. No. 10,028,747.
Application No. 15/861,482 is a continuation-in-part of application No. 14/526,600, filed on Oct. 29, 2014, now abandoned.
Application No. 15/081,909 is a continuation-in-part of application No. 14/526,600, filed on Oct. 29, 2014, now abandoned.
Application No. 15/080,915 is a continuation-in-part of application No. 14/526,600, filed on Oct. 29, 2014, now abandoned.
Application No. 15/080,915 is a continuation-in-part of application No. 12/989,048, filed on Oct. 21, 2010, now Pat. No. 8,974,487.
(63) Continuation-in-part of application No. 17/829,313, filed on May 31, 2022.
(63) Continuation-in-part of application No. 17/476,845, filed on Sep. 16, 2021.
(60) Provisional application No. 63/119,774, filed on Dec. 1, 2020.
(60) Provisional application No. 62/794,609, filed on Jan. 19, 2019.
(60) Provisional application No. 62/794,607, filed on Jan. 19, 2019.
(60) Provisional application No. 62/794,609, filed on Jan. 19, 2019.
(60) Provisional application No. 62/794,607, filed on Jan. 19, 2019.
(60) Provisional application No. 62/794,609, filed on Jan. 19, 2019.
(60) Provisional application No. 62/794,607, filed on Jan. 19, 2019.
(60) Provisional application No. 62/720,173, filed on Aug. 21, 2018.
(60) Provisional application No. 62/589,754, filed on Nov. 22, 2017.
(60) Provisional application No. 62/472,519, filed on Mar. 16, 2017.
(60) Provisional application No. 62/589,754, filed on Nov. 22, 2017.
(60) Provisional application No. 62/472,519, filed on Mar. 16, 2017.
(60) Provisional application No. 62/444,860, filed on Jan. 11, 2017.
(60) Provisional application No. 61/897,245, filed on Oct. 30, 2013.

(51) Int. Cl. A61B 17/12

(52) U.S. Cl. CPC A61B 17/12172; A61B 17/12113

(57) ABSTRACT

Disclosed herein is an intrasacular aneurysm occlusion device with a linearly-aligned proximal-to-distal stack of embolic structures which is configured to be inserted into an aneurysm sac and then radially-expanded and longitudinally-contracted. The stack can be shaped like a 3D revolution of three single phases (or one and half full phases) of a sine wave around its central longitudinal axis. There can be openings in the stack which allow insertion of embolic material (e.g. coils, hydrogels, or congealing material) into the embolic structures.

### CROSS-REFERENCE TO RELATED APPLICATIONS

This application is a continuation-in-part of U.S Pat. Application 17965502 filed on Oct. 10, 2013, a continuation-in-part of U.S Pat. Application 17829313 filed on May 31, 2022, and a continuation-in-part of U.S. Pat. Application 17476845 filed on Sept. 9, 2016.

U.S Pat. Application 17829313 was a continuation-in-part of U.S. Pat. Application 17485390 filed on Sept. 25, 2021, was a continuation-in-part of U.S. Pat. Application 17476845 filed on Sept. 16, 2021, was a continuation-in-part of U.S. Pat. Application 17472674 filed on Sept. 12, 2021, was a continuation-in-part of U.S. Pat. application 17467680 filed on Sept. 7, 2021, was a continuation-in-part of U.S. Pat. application 17466497 filed on Sept. 3, 2021, was a continuation-in-part of U.S. Pat. application 17353652 filed on Jun. 6, 2021, was a continuation-in-part of U.S. Pat. Application 17220002 filed on Apr. 1, 2021, was a continuation-in-part of U.S. Pat. Application 17214827 filed on Mar. 27, 2021, was a continuation-in-part of U.S. Pat. Application 17211446 filed on Mar. 24, 2021, was a continuation-in-part of U.S. Pat. application 16693267 filed on Nov. 23, 2019, and was a continuation-in-part of U.S. Pat. application 16660929 filed on Oct. 23, 2019.

U.S Pat. Application 17220002 was a continuation-in-part of U.S. Pat. Application 17214827 filed on Mar. 27, 2021. U.S Pat. Application 17220002 was a continuation-in-part of U.S. Pat. Application 17211446 filed on Mar. 24, 2021. U.S Pat. Application 17220002 claimed the priority benefit of U.S. Provisional Pat. Application 63119774 filed on Dec. 1, 2020. U.S Pat. Application 17220002 was a continuation-in-part of U.S. Pat. Application 16693267 filed on Nov. 23, 2019. U.S Pat. Application 17220002 was a continuation-in-part of U.S. Pat. Application 16660929 filed on Oct. 23, 2019.

U.S Pat. Application 16693267 was a continuation-in-part of U.S. Pat. Application 16660929 filed on Oct. 23, 2019. U.S Pat. Application 16693267 claimed the priority benefit of U.S. Provisional Pat. Application 62794609 filed on Jan. 19, 2019. U.S Pat. Application 16693267 claimed the priority benefit of U.S. Provisional Pat. Application 62794607 filed on Jan. 19, 2019. U.S Pat. Application 16693267 was a continuation-in-part of U.S. Pat. Application 16541241 filed on Aug. 15, 2019. U.S Pat. Application 16693267 was a continuation-in-part of U.S. Pat. Application 15865822 filed on Jan. 9, 2018 which issued as U.S. Pat. 10716573 on Jul. 21, 2020. U.S Pat. Application 16693267 was a continuation-in-part of U.S. Pat. application 15861482 filed on Jan. 3, 2018.

U.S Pat. Application 16660929 claimed the priority benefit of U.S. Provisional Pat. Application 62794609 filed on Jan. 19, 2019. U.S Pat. Application 16660929 claimed the priority benefit of U.S. Provisional Pat. Application 62794607 filed on Jan. 19, 2019. U.S Pat. Application 16660929 was a continuation-in-part of U.S. Pat. Application 16541241 filed on Aug. 15, 2019. U.S Pat. Application 16660929 was a continuation-in-part of U.S. Pat. Application 15865822 filed on Jan. 9, 2018 which issued as U.S. Pat. 10716573 on Jul. 21, 2020. U.S Pat. Application 16660929 was a continuation-in-part of U.S. Pat. Application 15861482 filed on Jan. 3, 2018.

U.S Pat. Application 16541241 claimed the priority benefit of U.S. Provisional Pat. Application 62794609 filed on Jan. 19, 2019. U.S Pat. Application 16541241 claimed the priority benefit of U.S. Provisional Pat. Application 62794607 filed on Jan. 19, 2019. U.S Pat. Application 16541241 claimed the priority benefit of U.S. Provisional Pat. Application 62720173 filed on Aug. 21, 2018. U.S Pat. Application 16541241 was a continuation-in-part of U.S. Pat. Application 15865822 filed on Jan. 9, 2018 which issued as U.S. Pat. 10716573 on Jul. 21, 2020

U.S Pat. Application 15865822 claimed the priority benefit of U.S. Provisional Pat. Application 62589754 filed on Nov. 22, 2017. U.S Pat. Application 15865822 claimed the priority benefit of U.S. Provisional Pat. Application 62472519 filed on Mar. 16, 2017. U.S Pat. Application 15865822 was a continuation-in-part of U.S. Pat. Application 15081909 filed on Mar. 27, 2016. U.S Pat. Application 15865822 was a continuation-in-part of U.S. Pat. Application 14526600 filed on Oct. 29, 2014.

U.S Pat. Application 15861482 claimed the priority benefit of U.S. Provisional Pat. Application 62589754 filed on Nov. 22, 2017. U.S Pat. Application 15861482 claimed the priority benefit of U.S. Provisional Pat. Application 62472519 filed on Mar. 3, 2016. U.S Pat. Application 15861482 claimed the priority benefit of U.S. Provisional Pat. Application 62444860 filed on Jan. 11, 2017. U.S Pat. Application 15861482 was a continuation-in-part of U.S. Pat. Application 15080915 filed on Mar. 25, 2016 which issued as U.S. Pat. 10028747 on Jul. 24, 2018. U.S Pat. Application 15861482 was a continuation-in-part of U.S. Pat. Application 14526600 filed on Oct. 29, 2014.

U.S Pat. Application 15081909 was a continuation-in-part of U.S. Pat. Application 14526600 filed on Oct. 29, 2014. U.S Pat. Application 15080915 was a continuation-in-part of U.S. Pat. Application 14526600 filed on Oct. 29, 2014. U.S Pat. Application 14526600 claimed the priority benefit of U.S. Provisional Pat. Application 61897245 filed on Oct. 30, 2013. U.S Pat. Application 14526600 was a continuation-in-part of U.S. Pat. Application 12989048 filed on Oct. 21, 2010 which issued as U.S. Pat. 8974487 on Mar. 10, 2015. U.S Pat. Application 12989048 claimed the priority benefit of U.S. Provisional patent application 61126047 filed on May 21, 2008. U.S Pat. Application 12989048 claimed the priority benefit of U.S. Provisional Pat. Application 61126027 filed on May 21, 2008.

The entire contents of these related applications are incorporated herein by reference.

### FEDERALLY SPONSORED RESEARCH

Not Applicable

### SEQUENCE LISTING OR PROGRAM

Not Applicable

### BACKGROUND -- FIELD OF INVENTION

This invention relates to devices and methods for occluding a cerebral aneurysm.

### INTRODUCTION

An aneurysm is an abnormal bulging of a blood vessel wall. The vessel from which the aneurysm protrudes is the parent vessel. Saccular aneurysms look like a sac protruding out from the parent vessel. Saccular aneurysms have a neck and can be prone to rupture. Fusiform aneurysms are a form of aneurysm in which a blood vessel is expanded circumferentially in all directions. Fusiform aneurysms generally do not have a neck and are less prone to rupturing than saccular aneurysms. As an aneurysm grows larger, its walls generally become thinner and weaker. This decrease in wall integrity, particularly for saccular aneurysms, increases the risk of the aneurysm rupturing and hemorrhaging blood into the surrounding tissue, with serious and potentially fatal health outcomes.

Cerebral aneurysms, also called brain aneurysms or intracranial aneurysms, are aneurysms that occur in the intercerebral arteries that supply blood to the brain. The majority of cerebral aneurysms form at the junction of arteries at the base of the brain that is known as the Circle of Willis where arteries come together and from which these arteries send branches to different areas of the brain. Although identification of intact aneurysms is increasing due to increased use of outpatient imaging such as outpatient MRI scanning, many cerebral aneurysms still remain undetected unless they rupture. If they do rupture, they often cause stroke, disability, and/or death. The prevalence of cerebral aneurysms is generally estimated to be in the range of 1%-5% of the general population or approximately 3-15 million people in the U.S. alone. Approximately 30,000 people per year suffer a ruptured cerebral aneurysm in the U.S. alone. Approximately one-third to one-half of people who suffer a ruptured cerebral aneurysm die within one month of the rupture. Sadly, even among those who survive, approximately one-half suffer significant and permanent deterioration of brain function. Better alternatives for cerebral aneurysm treatment are needed.

### Review of the Relevant Art

There has been considerable innovation concerning intrasacular devices to occlude cerebral aneurysms, including the following relevant art. U.S. Pat. Application Publication 20120283768 (Cox et al., Nov. 8, 2012, “Method and Apparatus for the Treatment of Large and Giant Vascular Defects”) discloses the deployment of multiple permeable shell devices within a single vascular defect. U.S. Pat. Application publications 20140330299 (Rosenbluth et al., Nov. 6, 2014, “Embolic Occlusion Device and Method”), 20180303486 (Rosenbluth et al., Oct. 25, 2018, “Embolic Occlusion Device and Method”), and 20210259699 (Rosenbluth et al., Aug. 26, 2021, “Embolic Occlusion Device and Method”) disclose an occlusion device with a tubular braided member having a first end and a second end and extending along a longitudinal axis, the tubular braided member having a repeating pattern of larger diameter portions and smaller diameter portions arrayed along the longitudinal axis.

U.S. Pat. Application Publication 20160249937 (Marchand et al., Sept. 1, 2016, “Multiple Layer Filamentary Devices for Treatment of Vascular Defects”) and U.S. Pat. 10238393 (Marchand et al., Mar. 26, 2019, “Multiple Layer Filamentary Devices for Treatment of Vascular Defects”) disclose a permeable shell and an inner structure configured to occlude blood flow. U.S. Pat. application publications 20160367260 (Hewitt et al., Dec. 22, 2016, “Devices for Therapeutic Vascular Procedures”), 20170128077 (Hewitt et al., May 5, 2017, “Devices for Therapeutic Vascular Procedures”), and 20190223881 (Hewitt et al., Jul. 25, 2019, “Devices for Therapeutic Vascular Procedures”) disclose a self-expanding resilient permeable shell made from elongate resilient filaments.

U.S. Pat. Application publications 20170156734 (Griffin, Jun. 8, 2017, “Occlusion Device”), 20190269414 (Griffin, Sept. 5, 2019, “Occlusion Device”), and 20220313274 (Griffin, Oct. 6, 2022, “Occlusion Device”) and U.S. Pat. 10285711 (Griffin, May 14, 2019, “Occlusion Device”) disclose a continuous compressible mesh structure comprising axial mesh carriages configured end to end, wherein each end of each carriage is a pinch point in the continuous mesh structure. U.S. Pat. Application Publications 20170224350 (Shimizu et al., Aug. 10, 2017, “Devices for Vascular Occlusion”), 20200323534 (Shimizu et al., Oct. 15, 2020, “Devices for Vascular Occlusion”), and U.S. Pat. Application Publication 20210228214 (Bowman et al., Jul. 29, 2021, “Devices for Vascular Occlusion”), and U.S. Pat. 10980545 (Bowman et al., Apr. 20, 2021, “Devices for Vascular Occlusion”) disclose an occlusive device, occlusive device delivery system, method of using, and method of delivering an occlusive device.

U.S. Pat. Application Publication 20170281194 (Divino et al., Oct. 5, 2017, “Embolic Medical Devices”) discloses an occlusive device with an elongate member having opposing first and second side edges which extend longitudinally along the member and a member width, wherein this member has a collapsed configuration in which the first and second side edges are curled toward each other about a longitudinal axis of the member. U.S. Pat. Application Publication 20190343532 (Divino et al., Nov. 14, 2019, “Occlusive Devices”) discloses a device with at least one expandable structure which is adapted to transition from a compressed configuration to an expanded configuration when released into an aneurysm. U.S. Pat. 10136896 (Hewitt et al., Nov. 27, 2018, “Filamentary Devices for Treatment of Vascular Defects”) and U.S. Pat. application publication 20220257260 (Hewitt et al., Aug. 18, 2022, “Filamentary Devices for Treatment of Vascular Defects”) disclose an implant with multiple mesh layers.

U.S. Pat. Application Publication 20200038034 (Maguire et al., Feb. 6, 2020, “Vessel Occluder”) discloses a vessel occluder with an expandable mesh portion having a flexible membrane that expands within a cavity of the expandable mesh portion. U.S. Pat. Application Publications 20200205841 (Aboytes et al., Jul. 2, 2020, “Devices, Systems, and Methods for the Treatment of Vascular Defects”), 20210244420 (Aboytes et al., Aug. 12, 2021, “Devices and Methods for the Treatment of Vascular Defects”), and 20210378681 (Aboytes et al., Dec. 9, 2021, “Devices, Systems, and Methods for the Treatment of Vascular Defects”) disclose aneurysm occlusion devices with a first configuration in which a first portion and a second portion are substantially linearly aligned and a second configuration in which the second portion at least partially overlaps the first portion.

U.S. Pat. Application Publication 20200289125 (Dholakia et al., Sept. 17, 2020, “Filamentary Devices Having a Flexible Joint for Treatment of Vascular Defects”) disclose an implant with a first permeable shell having a proximal end with a concave or recessed section and a second permeable shell having a convex section that mates with the concave or recessed section. U.S. Pat. Application Publication 20210282785 (Dholakia et al., Sept. 16, 2021, “Devices Having Multiple Permeable Shells for Treatment of Vascular Defects”) discloses a device with a plurality of permeable shells connected by a plurality of coils.

U.S. Pat. 11202636 (Zaidat et al., Dec. 21, 2021, “Systems and Methods for Treating Aneurysms”) and U.S. Pat. Application Publications 20220022884 (Wolfe et al., Jan. 27, 2022, “Systems and Methods for Treating Aneurysms”) and 20220211383 (Pereira et al., Jul. 7, 2022, “Systems and Methods for Treating Aneurysms”) disclose an apparatus for treating an aneurysm including an occlusion element configured to be releasably coupled to an elongate delivery shaft and a distal end, a proximal end, and a longitudinal axis extending between the distal end and the proximal end. U.S. Pat. Application Publication 20220087681 (Xu et al., Mar. 24, 2022, “Inverting Braided Aneurysm Implant with Dome Feature”) discloses an implant with a dome feature configured to press into aneurysm walls near the aneurysm’s dome and facilitate securement of the braid across the aneurysm’s neck.

### SUMMARY OF THE INVENTION

This invention is an intrasacular occlusion device with a proximal-to-distal stack of embolic structures. In an example, this invention can be embodied in an intrasacular aneurysm occlusion device with a linearly-aligned proximal-to-distal stack of three embolic structures which is configured to be inserted into an aneurysm sac and then radially-expanded and longitudinally-contracted. In an example, embolic structures in the stack of embolic structures can have ellipsoidal or oblate spheroidal shapes. In an example, the combined stack of embolic structures can be shaped like a 3D revolution of three single phases (or one and half full phases) of a sine wave around its central longitudinal axis. In an example, there can be openings in the stack which allow insertion of embolic material (e.g. coils, hydrogels, or congealing material) into the interiors of one or more of the embolic structures.

Each of the three embolic structures in this intrasacular stack serves a purpose. The proximal embolic structure serves as a neck bridge. The proximal embolic structure covers the neck of the aneurysm from inside the aneurysm sac and prevents blood flow from the parent vessel of the aneurysm into the aneurysm sac. The middle embolic structure serves as an anchor for the device. The middle embolic structure spans the largest diameter of the aneurysm sac and prevents the proximal embolic structure from slipping outward into the parent vessel. The distal embolic structure helps to seal the neck bridge. The distal embolic structure contacts the dome of the aneurysm sac and transmits pressure from this contact with the dome to the proximal embolic structure, pressing the proximal embolic structure against the inside of the aneurysm neck and preventing it from slipping inward into the main body of the aneurysm sac.

### BRIEF INTRODUCTION TO THE FIGURES

FIGS. 1 through 3 show three views, at three different times, of a stack of shape-changing ellipsoidal structures which are delivered through a catheter into an aneurysm sac.

FIG. 4 shows a stack of ellipsoidal embolic structures wherein a middle structure is wider than proximal and distal structures.

FIG. 5 shows a stack of ellipsoidal embolic structures wherein proximal and middle structures are wider than a distal structure.

FIG. 6 shows a stack of ellipsoidal embolic structures wherein proximal and distal structures are wider than a middle structure.

FIG. 7 shows a stack of embolic structures with ellipsoidal proximal and distal structures and a toroidal middle structure.

FIG. 8 shows a stack of embolic structures with an ellipsoidal middle structure and toroidal proximal and distal structures.

FIG. 9 shows a stack of toroidal embolic structures.

FIG. 10 shows a stack of embolic structures with ellipsoidal proximal and distal structures and a peanut-shaped middle structure.

FIG. 11 shows a stack of embolic structures with an ellipsoidal middle structure and peanut-shaped proximal and distal structures.

FIG. 12 shows a stack of embolic structures with toroidal proximal and distal structures and a peanut-shaped middle structure.

FIG. 13 shows a stack of embolic structures with ellipsoidal proximal and distal structures and a tree ornament shaped middle structure.

FIG. 14 shows a stack of embolic structures with tree ornament shaped proximal and distal structures and an ellipsoidal structure.

FIG. 15 shows a stack of tree ornament shaped embolic structures.

FIG. 16 shows a stack of embolic structures with ellipsoidal proximal and distal structures and a cardioid shaped middle structure.

FIG. 17 shows a stack of embolic structures with cardioid shaped proximal and distal structures and an ellipsoidal middle structure.

FIG. 18 shows a stack of cardioid shaped embolic structures.

FIG. 19 shows a stack of embolic structures wherein a middle structure has high flexibility, high elasticity, and/or low durometer.

FIG. 20 shows a stack of embolic structures wherein a distal structure has high flexibility, high elasticity, and/or low durometer.

FIG. 21 shows a stack of embolic structures wherein proximal and distal structures have high flexibility, high elasticity, and/or low durometer.

FIG. 22 shows a stack of embolic structures wherein a proximal structure is filled with embolic material.

FIG. 23 shows a stack of embolic structures wherein a middle structure is filled with embolic material.

FIG. 24 shows a stack of embolic structures wherein a distal structure is filled with embolic material.

FIG. 25 shows a stack of embolic structures wherein proximal and middle structures are filled with embolic material.

FIG. 26 shows a stack of embolic structures wherein middle and distal structures are filled with embolic material.

FIG. 27 shows a stack of embolic structures which are all filled with embolic material.

FIG. 28 shows a stack of embolic structures wherein embolic material is inserted from a catheter into middle and distal structures.

FIG. 29 shows a stack of embolic structures wherein embolic material is inserted from a catheter into all of the structures.

FIGS. 30 through 32 show three configurations of a stack of embolic structures whose shape is changed by a device operator pulling or pushing a wire.

FIGS. 33 through 36 show four configurations of a stack of centrally-aligned embolic structures which is created by radially-constraining a tubular mesh.

### DETAILED DESCRIPTION OF THE FIGURES

In an example, an intrasacular aneurysm occlusion device with a stack of embolic structures can comprise: a proximal embolic structure which is configured to be deployed in an aneurysm sac at a first distance from the aneurysm neck; a middle embolic structure which is configured to be deployed in the aneurysm sac at a second distance from the aneurysm neck; and a distal embolic structure which is configured to be deployed in the aneurysm sac at a third distance from the aneurysm neck; wherein the second distance is greater than the first distance; wherein the third distance is greater than the second distance; and wherein the centers of the proximal embolic structure, the middle embolic structure, and the distal embolic structure are linearly-aligned.

This intrasacular stack of embolic structures can also be described as having three embolic structures: a proximal embolic structure which is closest to the aneurysm neck when deployed in the aneurysm sac; a distal embolic structure which is farthest from the aneurysm neck when deployed in the aneurysm sac, and a middle embolic structure between the proximal embolic structure and the distal embolic structure. The distance between an embolic structure and the aneurysm neck can be defined as the distance between two parallel planes: a first virtual plane which passes through the centroid of the embolic structure; and a second virtual plane which passes through the narrowest perimeter of the aneurysm neck.

Each of three embolic structures in an intrasacular stack of embolic structures can serve a purpose. The proximal embolic structure can serve as a neck bridge. The proximal embolic structure covers the neck of the aneurysm from inside the aneurysm sac and prevents blood flow from the parent vessel of the aneurysm into the aneurysm sac. The middle embolic structure serves as an anchor for the device. The middle embolic structure can span the largest diameter of the aneurysm sac and prevent the proximal embolic structure from slipping outward into the parent vessel. The distal embolic structure can help to seal the neck bridge. The distal embolic structure can contact the dome of the aneurysm sac and transmit pressure from this contact with the dome to the proximal embolic structure, pressing the proximal embolic structure against the inside of the aneurysm neck and preventing it from slipping inward into the main body of the aneurysm sac.

In an example, proximal, middle, and distal embolic structures in a stack of embolic structures can be created separately and then connected and/or attached together. In an example, each of the embolic structures can be a mesh, net, braid, and/or stent. In an example, an embolic structure can be made from a metal, a polymer, or a combination of a metal and a polymer. In an example, proximal, middle, and distal embolic structures in a stack can be connected by a longitudinal wire, cord, string, strand, tube, catheter, or coil. In an example, a longitudinal wire, cord, string, strand, tube, catheter, or coil can pass through the centers of proximal, middle, and distal embolic structures. In an example, proximal, middle, and distal embolic structures can be contiguous with each other (e.g. touching each other) after they are connected together in a stack. Alternatively, there can be (small) gaps between proximal, middle, and distal embolic structures in a stack.

In an example, proximal, middle, and distal embolic structures can be connected to each other by adhesion, gluing, melting, and/or soldering. In an example, proximal, middle, and distal embolic structures can be connected to each other by weaving, braiding, or sewing. In an example, proximal, middle, and distal embolic structures can be connected to each other by pinching, binding, clipping, hooking, or riveting. In an example, proximal, middle, and distal embolic structures can be tied to each other. In an example, proximal, middle, and distal embolic structures can be connected to each other by pinching or compressing their ends in bands, rings, or cylinders. In an example, proximal, middle, and distal embolic structures can be connected to each other by pinching or compressing their ends between concentric bands, rings, or cylinders.

In another example, proximal, middle, and distal embolic structures can be made from a single continuous structure. In an example, proximal, middle, and distal embolic structures can be lobes, bulges, undulations, or segments which are created from a single continuous structure. In an example, proximal, middle, and distal embolic structures can be formed by radially-constraining segments of a tubular mesh at multiple locations. In an example, proximal, middle, and distal embolic structures in a stack of embolic structures can be a longitudinal series of lobes or bulges which are formed by radially-constraining, inverting, and/or everting a flexible mesh tube. In an example, proximal, middle, and distal embolic structures in a stack of embolic structures can be a longitudinal series of lobes or bulges which are formed by radially-constraining a flexible tube with bands, rings, or cylinders at multiple locations along its longitudinal axis. In an example, proximal, middle, and distal embolic structures can be formed by radially-constraining and then inverting a tubular mesh. In an example, proximal, middle, and distal embolic structures can be formed by radially-constraining and then everting a tubular mesh.

In an example, proximal, middle, and distal embolic structures in a stack of embolic structures can have longitudinally-expanded and radially-compressed configurations as they are delivered through a catheter to an aneurysm sac. In an example, these embolic structures can shrink longitudinally and expand radially after they exit a catheter in an aneurysm sac.

In an example, embolic structures can have a first configuration as they travel through a catheter and a second configuration after they expand in an aneurysm sac, wherein the orientation of their longest axis in the second configuration is perpendicular to the orientation of their longest axis in the first configuration. In an example, embolic structures in a stack of embolic structures can be inserted and expanded sequentially within an aneurysm sac, wherein one embolic structure is inserted and expanded before the next embolic structure is inserted into the aneurysm sac. In an example, a plurality of embolic structures in a stack of embolic structures can be inserted and expanded within an aneurysm sac at the same time.

In an example, proximal, middle, and distal embolic structures in a stack of embolic structures can self-expand radially after they exit a catheter in an aneurysm sac. Alternatively, the radial expansion of an embolic structure can be controlled by an operator of the device. In an example, a device operator can control the amount, direction, and/or timing of radial expansion of an embolic structure by a mechanism selected from the group consisting of: pulling a wire or string; applying electromagnetic energy; and rotating a wire or catheter. In an example, a device operator can selectively control the amount, direction, and/or timing of radial expansion of each embolic structure by a mechanism selected from the group consisting of: pulling a wire or string; applying electromagnetic energy; and rotating a wire or catheter. This can enable the device operator to customize the expanded shape of the stack to conform to the shape and size of an irregularly-shape aneurysm sac.

A proximal, middle, or distal embolic structure can have a first shape while it is being delivered through a catheter to an aneurysm sac and a second shape after it has been inserted and self-expanded within the aneurysm sac. In an example, the second shape of an embolic structure can be selected from the group consisting of: spherical shape, ellipsoidal shape, oblate spheroid shape, barrel shape, canister shape, racetrack ovaloid shape, disk shape, apple shape, pumpkin shape, toroidal shape, doughnut shape, tree ornament (3D revolved sine-wave), egg shape, pear shape, tear-drop shape, cardioid shape, bullet shape, hemispherical shape, bowl shape, half-canister shape, funnel shape, jellyfish shape, 3D revolved horseshoe shape, jug or bottle shape, mushroom shape, hour-glass shape, hyperboloidal shape, peanut shape, dumbbell shape, reflected mushroom shape, Saturn shape, paper lantern shape, and geodesic shape. In an example, an embolic structure can be further expanded to a third shape by being filed with embolic material. In an example, the third shape can be an irregular shape which conforms to the walls of an irregularly-shaped aneurysm sac.

In an example, one of the embolic structure sin a stack can have an ellipsoidal, oblate spheroid, or ring shape and other embolic structures in the stack can have toroidal, doughnut, or peanut shapes. In an example, the shape of one or more embolic structures in a stack of embolic structures can be adjusted, controlled, and/or varied in real time by a device operator in order to best match the contours of a specific aneurysm sac. In an example, the shape of one or more embolic structures in a stack of embolic structures can be adjusted, controlled, and/or varied in real time by a device operator by a mechanism selected from the group consisting of: pulling a wire, cord, or string; applying electromagnetic energy; and rotating a wire or catheter. In an example, a device operator can control the post-expansion diameter of a proximal, middle, or distal embolic member by one or more of these mechanisms.

Many of these above-referenced shapes are commonly used and generally well-defined. However, some are less common. We now provide additional descriptions and/or definitions for the less-common shapes to clarify how they are used herein to define invention embodiments. An ellipsoid is a three-dimensional surface whose cross sections are all either ellipses or circles. An oblate spheroid is an ellipsoid created by compressing a sphere along one axis. A barrel shape can be described as a generally-cylindrical prism whose lower and upper circumferences are smaller than its mid-section circumference. A canister shape can be described as a generally-cylindrical prism, wherein the perimeter edges of its lower and upper surfaces are rounded. A racetrack ovaloid can be described is a generally-oval shape wherein at least half of the lengths of its longitudinal sides are straight and parallel to each other.

An apple shape can be described as sphere or oblate spheroid with a concave (e.g. funnel-shaped) indentation which is centrally located on its upper surface and a concave (e.g. funnel-shaped) indentation which is centrally located on its lower surface. A pumpkin shape is similar to an apple shape except that the concave indentations of a pumpkin shape are larger than those of an apple shape and its ratio of width to height is larger than that of an apple shape. A pumpkin shape can also be described as a surface of revolution which is formed by revolving between 60% and 80% of a circle or ellipse in three dimensions around an axis that is coplanar with the circle or ellipse.

A torus is defined herein as a surface of revolution which is formed by revolving a circle or ellipse in three dimensions around an axis that is coplanar with the circle or ellipse. Unless otherwise specified, it is assumed herein that the axis does not intersect the circle or ellipse, thereby creating a central opening in the torus (like a doughnut shape). A tree ornament shape can be described as an ellipsoid with an upward-facing spire and a downward-facing spire. Alternatively, a tree ornament shape can be described as a 3D-revolution of a single phase of a vertical sine wave around an axis which connects its open ends. As used herein, a cardioid and/or heart shape is the 3D revolution of a cardioid or heart shape. A cardioid is a curve which is traced by a point on the perimeter of a circle that is rolled around a fixed circle of the same radius. A canister shape can be described as a generally-cylindrical, wherein edge perimeter edges of its lower and upper surfaces are rounded. A half-canister shape is created by cutting the canister in half laterally.

The jellyfish shape discussed herein is formed by inverting one side of a hollow sphere or ellipsoid mesh into the other side of the sphere or ellipsoid mesh, but stopping the inversion action before the originally-polar ends of the sphere or ellipsoid contact each other. In an example, the post-inversion distance between the originally-polar ends can be between 5% and 35% of the pre-invention distance between them. A 3D-revolved horseshoe shape is created by revolving a horseshoe shape in three dimensions around the central longitudinal axis of the horseshoe.

A jug and/or bottle shape can be described as a shape with a generally-cylindrical lower portion and a funnel-shaped upper portion, wherein the maximum diameter of the upper portion is the diameter of the top of the lower portion. Alternatively, a jug and/or bottle shape can be described as a shape with a generally-cylindrical main body and an upwardly-tapered neck above the main body. Also, a diameter-to-neck ratio can be defined as: the maximum diameter of the cylindrical lower portion divided by the minimum diameter of the neck portion. If one wishes to distinguish between a bottle shape and a jug shape, a jug shape can be defined as having a diameter-to-neck ratio of 5 or greater and a bottle shape can be defined as a having a diameter-to-neck ratio between 2 and 5. An inverted jug and/or bottle shape is a vertically-inverted version of a jug and/or bottle shape.

A peanut shape can be described as two globular lobes which are connected by a narrower arcuate mid-section. A peanut shape can also be described the 3D-revolution of two parallel and overlapping, but not perfectly aligned, lemniscate figures around a longitudinal axis. A dumbbell shape can be described as two globular lobes or disks which are connected by a generally-cylindrical narrower column. A “reflected mushroom” or “double mushroom” shape can be created by combining first and second mushroom shapes, wherein the second mushroom is a reflection (around a base horizontal line) of the first mushroom. A Saturn shape can be described as sphere or ellipsoid with a ring and/or torus shaped protrusion around its central circumference. A paper lantern shape can be described as a barrel or canister shape with undulating (e.g. sinusoidal, zigzag, and/or pleated) longitudinal sides.

In an example, proximal, middle, and distal embolic structures in an intrasacular stack can all have the same shape. In an example, proximal, middle, and distal embolic structures can have different shapes. In an example, the shapes of each of the proximal, middle, and/or distal embolic structures can be individually and differentially selected from the group consisting of: spherical shape, ellipsoidal shape, oblate spheroid shape, barrel shape, canister shape, racetrack ovaloid shape, disk shape, apple shape, pumpkin shape, toroidal shape, doughnut shape, tree ornament (3D revolved sine-wave), egg shape, pear shape, tear-drop shape, cardioid shape, bullet shape, hemispherical shape, bowl shape, half-canister shape, funnel shape, jellyfish shape, 3D revolved horseshoe shape, jug or bottle shape, mushroom shape, hour-glass shape, hyperboloidal shape, peanut shape, dumbbell shape, reflected mushroom shape, Saturn shape, paper lantern shape, and geodesic shape.

In an example, a one or more embolic structures in a stack can have ellipsoidal, oblate spheroid, or ring shape and one or more of the other embolic structures in the stack can have a toroidal, doughnut, or peanut shape. In an example, proximal, middle, and distal embolic structures can all be same tree ornament shape and be the same size, wherein they collectively comprise a stack with a shape which is a 3D revolution of (three single phases or one-and-a-half full phases) of a sine wave around its central longitudinal axis. In an example, the amplitude and frequency of the sine wave in such a combined structure can be adjusted, controlled, and/or varied by a device operator in order to best match the contours of a specific aneurysm sac. In an example, with operator control of a 3D sine wave revolved shape, an operator can adjust the amplitude and frequency of the 3D sine wave shape.

In an example, a distal convexity of a first embolic structure can be nested inside a proximal concavity of a second embolic structure in a stack of embolic structures, wherein first embolic structure is proximal relative to the second embolic structure. In an example, a distal convexity of a proximal embolic structure can be nested inside a proximal concavity of a middle embolic structure in a stack of three embolic structures. In an example, a distal convexity of a middle embolic structure can be nested inside a proximal concavity of a distal embolic structure in a stack of three embolic structures. In an example, a distal concavity of a first embolic structure can be nested inside a proximal convexity of a second embolic structure in a stack of embolic structures, wherein first embolic structure is proximal relative to the second embolic structure. In an example, a distal concavity of a proximal embolic structure can be nested inside a proximal convexity of a middle embolic structure in a stack of three embolic structures. In an example, a distal concavity of a middle embolic structure can be nested inside a proximal convexity of a distal embolic structure in a stack of three embolic structures.

In an example, embolic structures in a stack of embolic structures can all be the same size. In an example, embolic structures in a stack can all have the same width and/or diameter. In an example, proximal embolic structures in an intrasacular stack of structures can be larger (e.g. wider and/or longer) than distal embolic structures in the stack. In an example, distal embolic structures in an intrasacular stack of structures can be larger (e.g. wider and/or longer) than proximal embolic structures in the stack. In an example, in a series of embolic structures which are sequentially inserted into an aneurysm sac, embolic structures which are inserted first can be smaller (e.g. narrower) and structures which are inserted later can be larger (e.g. wider). In an example, in a series of embolic structures which are sequentially inserted into an aneurysm sac, embolic structures which are inserted first can be smaller (e.g. narrower), structures which are inserted next can be larger (e.g. wider) and structures which are inserted last can be largest (e.g. widest).

In an example, a middle embolic structure in an intrasacular stack of three embolic structures can be wider (e.g. 25% to 200% wider) than each of a proximal embolic structure and a distal embolic structure. This design can help to keep the structure in the aneurysm sac if the structure engages the walls of the aneurysm sac at its widest section (e.g. if the middle section of the sac is widest). This can be optimal for berry aneurysms. In an example, a proximal embolic structure can be wider (e.g. 25% to 200% wider) than each of a middle embolic structure and a distal embolic structure. This design can help to maximize coverage of the aneurysm neck to minimize flow of blood into the aneurysm sac. This design can also be optimal for fusiform aneurysms. In an example, proximal and distal embolic structures can each be wider (e.g. 25% to 200% wider) than a middle embolic structure. This design can help the device to bend laterally, which can be help to occlude asymmetric and/or irregularly-shaped aneurysms. In an example, the size of one or more embolic structures in a stack of embolic structures can be adjusted, controlled, and/or varied in real time by a device operator in order to best match the contours of a specific aneurysm sac.

In an example, embolic structures in a stack can all have the same level of flexibility, elasticity, and/or durometer. In an example, a middle embolic structure can have greater flexibility, greater elasticity, and/or lower durometer than each of a proximal embolic structure and a distal embolic structure. In an example, a distal embolic structure can have greater flexibility, greater elasticity, and/or lower durometer than each of a proximal embolic structure and a middle embolic structure. In an example, proximal and distal embolic structures can have greater flexibility, greater elasticity, and/or lower durometer than a middle embolic structure. In an example, embolic structures in a stack can all have the same porosity level. In an example, a middle embolic structure can have greater porosity than each of a proximal embolic structure and a distal embolic structure. In an example, a distal embolic structure can have greater porosity than each of a proximal embolic structure and a middle embolic structure.

In an example, some or all of the interiors of one or more embolic structures in a stack can be filled with embolic members and/or material after the one or more embolic structures have been inserted into an aneurysm sac. In an example, embolic members and/or material can be selected from the group consisting of: embolic coils, hydrogels, microsponges, beads, ribbons, string-of-pearls strands of embolic components, and congealing material. In an example, an embolic structure can self-expand from a first shape to a second shape after it exits a catheter in an aneurysm sac. In an example, an embolic structure can further expand from the second shape to a third shape as the interior of the embolic member is filled with embolic material. The third shape can better conform to the walls of an irregularly-shaped aneurysm sac and thus reduce the risk of recanalization.

In an example, there can be an opening (or hole) in an embolic structure. In an example, this opening can be centrally and/or axially located on the proximal surface of an embolic structure. In an example, this opening can be non-centrally and/or off-axially located on the proximal surface of an embolic structure. In an example, embolic members and/or material can be inserted through this opening into the interior of the embolic structure. In an example, embolic members and/or material can be inserted through this opening into the interiors of other, more-distal, embolic structures in a stack. In an example, all three embolic structures in a stack can be filled with embolic members and/or material. In an example, only one or two of three embolic structures in a stack may be filled with embolic members and/or material. In an example, there can be a tube, catheter, and/or channel through the center of a first embolic structure through which embolic material travels into the interior of more-distal second embolic structure, but not into the rest of the interior of the first embolic structure.

In an example, there can also be a valve which opens or closes the opening (or hole) in an embolic structure. In an example, this valve enables embolic members and/or material to enter, but not exit, the embolic structure. In an example, this valve can be controlled remotely by the device operator. In an example, a valve can be remotely opened and/or closed by an operator by the application of electromagnetic energy. In an example, a valve can be remotely opened and/or closed by an operator by pulling a filament. In an example, a valve can be remotely opened and/or closed by an operator by pushing, pulling, or rotating a wire. In an example, a valve can be remotely opened and/or closed by an operator by cutting, pulling, or pushing a flap or plug. In an example, a valve can passively open when embolic members and/or material is pushed through it and can passively close afterward. In an example, a valve can be a leaflet valve.

In an example, embolic structures in a stack of embolic structures can be pushed out of a catheter into an aneurysm sac by movement of a wire, inner catheter, or plunger. In an example, embolic structures in a stack of embolic structures can be moved out of a catheter into an aneurysm sac by microscale motor (e.g. a conveyer-belt MEMS). In an example, embolic structures in a stack of embolic structures can be moved out of a catheter into an aneurysm sac by a rotating helically-threaded mechanism (e.g. an Archimedes screw mechanism). In an example, embolic structures in a stack of embolic structures can be pushed out of a catheter by fluid pressure. In an example, embolic structures in a stack of embolic structures can be moved out a catheter in a flow of liquid (e.g. a saline solution).

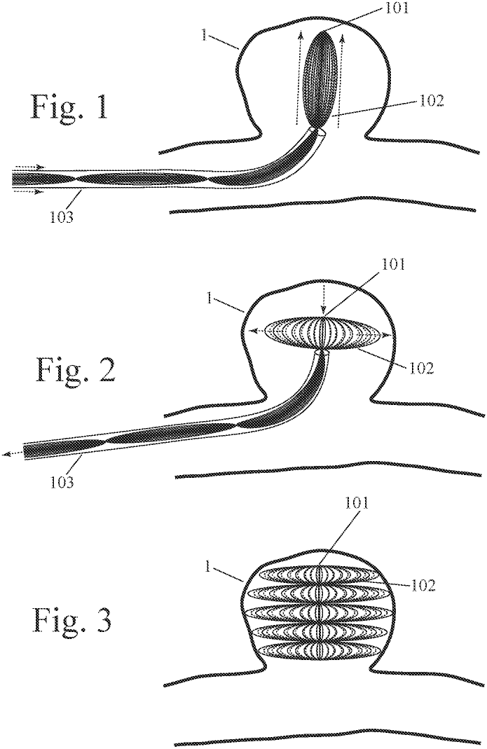

FIGS. 1 through 3 show three views, at three different times, of an intrasacular aneurysm occlusion device with a stack of embolic structures. This device comprises: (a) a longitudinal catheter that is configured to be inserted into a blood vessel, wherein this catheter has a longitudinal axis spanning from its proximal end to its distal end and wherein the distal end is first inserted into the blood vessel; and (b) a plurality of longitudinally-linked configuration-changing embolic structures which are configured to travel through the longitudinal catheter and to be inserted into an aneurysm; wherein each shape-changing embolic structure has its own internally-referenced Z axis, X axis, and Y axis; wherein its Z axis is substantially parallel to the longitudinal axis of the longitudinal catheter as the embolic structure travels through the longitudinal catheter, its X axis is substantially perpendicular to its Z axis, and its Y axis is substantially perpendicular to both its Z axis and X axis; wherein each configuration-changing embolic structure has a first configuration as the member travels through the longitudinal catheter and a second configuration within the aneurysm after it exits the longitudinal catheter; wherein the distance of the embolic structure spanning its Z axis is greater than the distance of the embolic structure spanning its X axis or Y axis in the first configuration; wherein the distance of the embolic structure spanning its Z axis is less than the distance of the embolic structure spanning its X axis or Y axis in the second configuration; wherein the cross-sectional shape of the embolic structure in an X-Z plane is substantially elliptical, oval, or another arcuate non-circular shape in the first configuration, with the longer dimension of the ellipse, oval, or another arcuate non-circular shape being along its Z axis; and wherein the cross-sectional shape of the embolic structure in the X-Z plane is substantially elliptical, oval, or another arcuate non-circulate shape in the second configuration, with the longer dimension of the ellipse, oval, or another arcuate non-circular shape being along its X axis.

FIGS. 1 through 3 can also be described as showing an intrasacular aneurysm occlusion device with a stack of embolic structures comprising: (a) a longitudinal catheter that is configured to be inserted into a blood vessel, wherein this blood vessel is the parent vessel from which an aneurysm has formed; and (b) a series of connected embolic ellipsoids, wherein these embolic ellipsoids are configured to travel in series through the longitudinal catheter and to be inserted into the aneurysm sac; wherein an embolic ellipsoid has a first orientation as it travels through the longitudinal catheter; wherein an embolic ellipsoid has a second orientation after it exits the longitudinal catheter; wherein in the first orientation the longitudinal axis of the ellipsoid is substantively parallel to the longitudinal axis of the longitudinal catheter; wherein in the second orientation the longitudinal axis of the ellipsoid is substantially perpendicular to its prior orientation traveling through the longitudinal catheter.

As shown in FIG. 2, each embolic ellipsoid (including 102) can have a first orientation as it travels through catheter 103 and a second orientation after it exits catheter 103 inside aneurysm sac 1. In an example, each embolic ellipsoid can have a longitudinal axis such as 101 for embolic ellipsoid 102. In an example, in the first orientation, the longitudinal axis (such as 101) of the ellipsoid (such as 102) can be substantively-parallel to the longitudinal axis of catheter 103. In an example, in the second orientation, the longitudinal axis (such as 101) of the ellipsoid (such as 102) can be substantially-perpendicular to its prior orientation traveling through catheter 103.

In an example, an embolic ellipsoid (such as 102) can be oriented as it travels through catheter 103 such that its longest axis is substantially-parallel to the longitudinal axis of catheter 103. In an example, an embolic ellipsoid (such as 102) can be compressed and/or reoriented after it exits catheter 103 so that its longest axis becomes substantially-parallel to the plane that is defined by the central circumference of the aneurysm neck. In an example, the longitudinal axes (such as 101) of the embolic ellipsoids (such as 102) as these ellipsoids travel through catheter 103 can become the virtual lateral axes (still 101) of these embolic ellipsoids (such as 102) when these ellipsoids are compressed and/or reoriented after they exit catheter 103.

In example, the longitudinal axes (including 102) of these ellipsoids (including 102) can be compressed after the ellipsoids exit catheter 103. In an example, this compression can be caused by movement of a wire, fiber, or other longitudinal flexible member that is connected to the ellipsoids. In an example, this compression can be caused by contact between the aneurysm wall and the ellipsoids. In an example, the embolic ellipsoids (including 102) can have a shape memory and a prior shape to which they return after their release from catheter 103. In an example, their return to a prior shape can cause the change in their orientation and/or compression after they exit catheter 103. In this example, the embolic ellipsoids (including 102) are wire structures. Example variations discussed elsewhere in this disclosure or priority-linked disclosures can also be applied to this example where relevant.

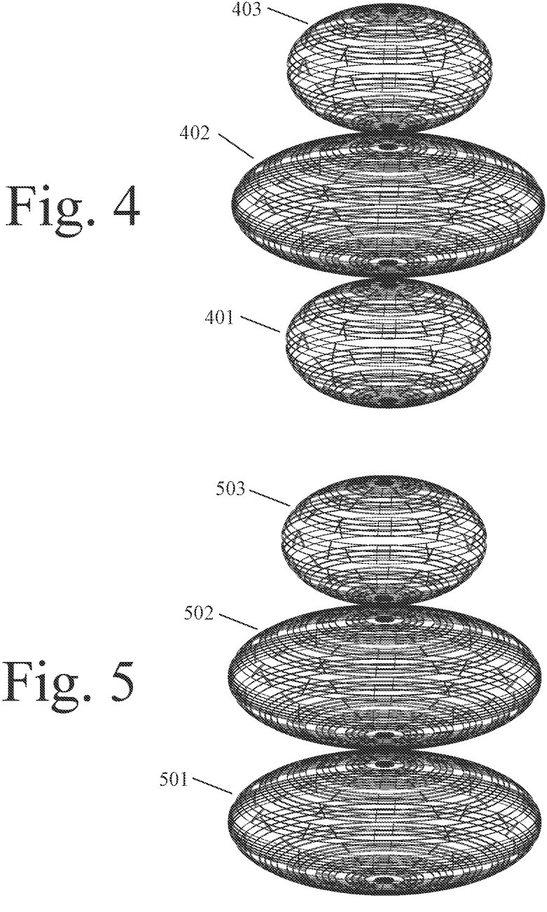

FIG. 4 shows an intrasacular aneurysm occlusion device with a stack of embolic structures comprising: a proximal embolic structure 401 which is configured to be in an aneurysm sac a first distance from the aneurysm neck; a middle embolic structure 402 which is configured to be in the aneurysm sac a second distance from the aneurysm neck; and a distal embolic structure 403 which is configured to be in the aneurysm sac a third distance from the aneurysm neck; wherein the second distance is greater than the first distance; wherein the third distance is greater than the second distance; wherein central axes of the proximal embolic structure, the middle embolic structure, and the distal embolic structure are linearly aligned; wherein the proximal embolic structure, the middle embolic structure, and the distal embolic structure have shapes selected from the group consisting of ellipsoid, oblate spheroid, and ovaloid; and wherein the middle embolic structure is between 25% and 250% wider than each of the proximal embolic structure and the distal embolic structure. Example variations discussed elsewhere in this disclosure or priority-linked disclosures can also be applied to this example where relevant.

FIG. 5 shows an intrasacular aneurysm occlusion device with a stack of embolic structures comprising: a proximal embolic structure 501 which is configured to be in an aneurysm sac a first distance from the aneurysm neck; a middle embolic structure 502 which is configured to be in the aneurysm sac a second distance from the aneurysm neck; and a distal embolic structure 503 which is configured to be in the aneurysm sac a third distance from the aneurysm neck; wherein the second distance is greater than the first distance; wherein the third distance is greater than the second distance; wherein central axes of the proximal embolic structure, the middle embolic structure, and the distal embolic structure are linearly aligned; wherein the proximal embolic structure, the middle embolic structure, and the distal embolic structure have shapes selected from the group consisting of ellipsoid, oblate spheroid, and ovaloid; and wherein the proximal and middle embolic structures are between 25% and 250% wider than the distal embolic structure. Example variations discussed elsewhere in this disclosure or priority-linked disclosures can also be applied to this example where relevant.

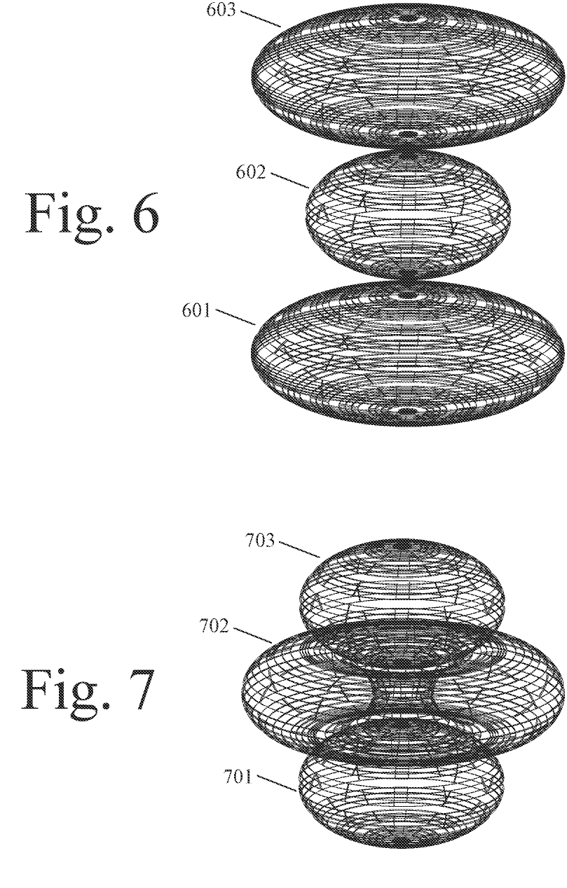

FIG. 6 shows an intrasacular aneurysm occlusion device with a stack of embolic structures comprising: a proximal embolic structure 601 which is configured to be in an aneurysm sac a first distance from the aneurysm neck; a middle embolic structure 602 which is configured to be in the aneurysm sac a second distance from the aneurysm neck; and a distal embolic structure 603 which is configured to be in the aneurysm sac a third distance from the aneurysm neck; wherein the second distance is greater than the first distance; wherein the third distance is greater than the second distance; wherein central axes of the proximal embolic structure, the middle embolic structure, and the distal embolic structure are linearly aligned; wherein the proximal embolic structure, the middle embolic structure, and the distal embolic structure have shapes selected from the group consisting of ellipsoid, oblate spheroid, and ovaloid; and wherein the proximal embolic structure and the distal embolic structure are between 25% and 250% wider than the middle embolic structure. Example variations discussed elsewhere in this disclosure or priority-linked disclosures can also be applied to this example where relevant.

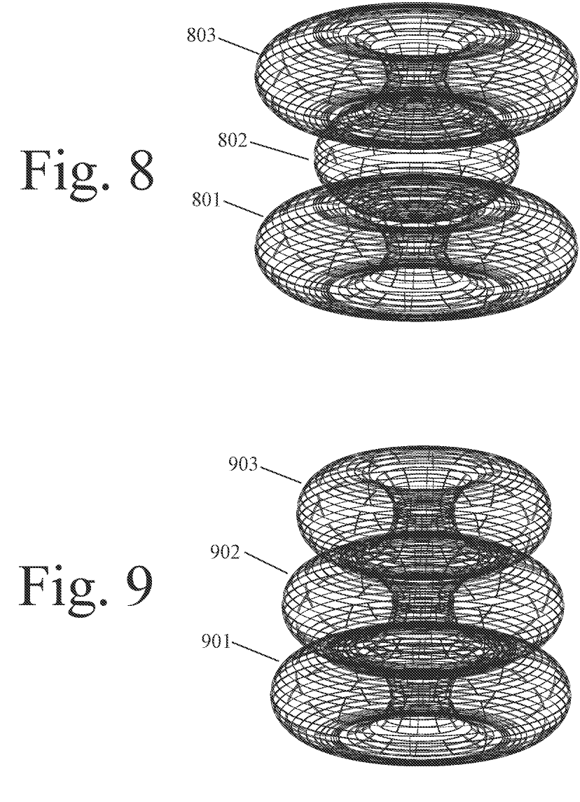

FIG. 7 shows an intrasacular aneurysm occlusion device with a stack of embolic structures comprising: a proximal embolic structure 701 which is configured to be in an aneurysm sac a first distance from the aneurysm neck; a middle embolic structure 702 which is configured to be in the aneurysm sac a second distance from the aneurysm neck; and a distal embolic structure 703 which is configured to be in the aneurysm sac a third distance from the aneurysm neck; wherein the second distance is greater than the first distance; wherein the third distance is greater than the second distance; wherein central axes of the proximal embolic structure, the middle embolic structure, and the distal embolic structure are linearly aligned; wherein the proximal embolic structure and the distal embolic structure have shapes selected from the group consisting of ellipsoid, oblate spheroid, and ovaloid; and wherein the middle embolic structure has a shape selected from the group consisting of torus and doughnut. In this example: an arcuate proximal surface of the ellipsoidal distal embolic structure is nested in the arcuate distal surface of the toroidal middle embolic structure; and an arcuate distal surface of the ellipsoidal proximal embolic structure is nested in an arcuate proximal surface of the toroidal middle embolic structure. Example variations discussed elsewhere in this disclosure or priority-linked disclosures can also be applied to this example where relevant.

FIG. 8 shows an intrasacular aneurysm occlusion device with a stack of embolic structures comprising: a proximal embolic structure 801 which is configured to be in an aneurysm sac a first distance from the aneurysm neck; a middle embolic structure 802 which is configured to be in the aneurysm sac a second distance from the aneurysm neck; and a distal embolic structure 803 which is configured to be in the aneurysm sac a third distance from the aneurysm neck; wherein the second distance is greater than the first distance; wherein the third distance is greater than the second distance; wherein central axes of the proximal embolic structure, the middle embolic structure, and the distal embolic structure are linearly aligned; wherein the middle embolic structure has a shape selected from the group consisting of ellipsoid, oblate spheroid, and ovaloid; and wherein the proximal embolic structure and the distal embolic structure have shapes selected from the group consisting of torus and doughnut. In this example: an arcuate distal surface of the ellipsoidal middle embolic structure is nested in an arcuate proximal surface of the toroidal distal embolic structure; and an arcuate proximal surface of the ellipsoidal middle embolic structure is nested in an arcuate distal surface of the toroidal proximal embolic structure. Example variations discussed elsewhere in this disclosure or priority-linked disclosures can also be applied to this example where relevant.

FIG. 9 shows an intrasacular aneurysm occlusion device with a stack of embolic structures comprising: a proximal embolic structure 901 which is configured to be in an aneurysm sac a first distance from the aneurysm neck; a middle embolic structure 902 which is configured to be in the aneurysm sac a second distance from the aneurysm neck; and a distal embolic structure 903 which is configured to be in the aneurysm sac a third distance from the aneurysm neck; wherein the second distance is greater than the first distance; wherein the third distance is greater than the second distance; wherein central axes of the proximal embolic structure, the middle embolic structure, and the distal embolic structure are linearly aligned; wherein the proximal, middle, and distal embolic structures have toroidal or doughnut shapes. In an example, embolic material can be inserted into the aneurysm sac through the central holes in the toroidal proximal, middle, and distal embolic structures. Example variations discussed elsewhere in this disclosure or priority-linked disclosures can also be applied to this example where relevant.

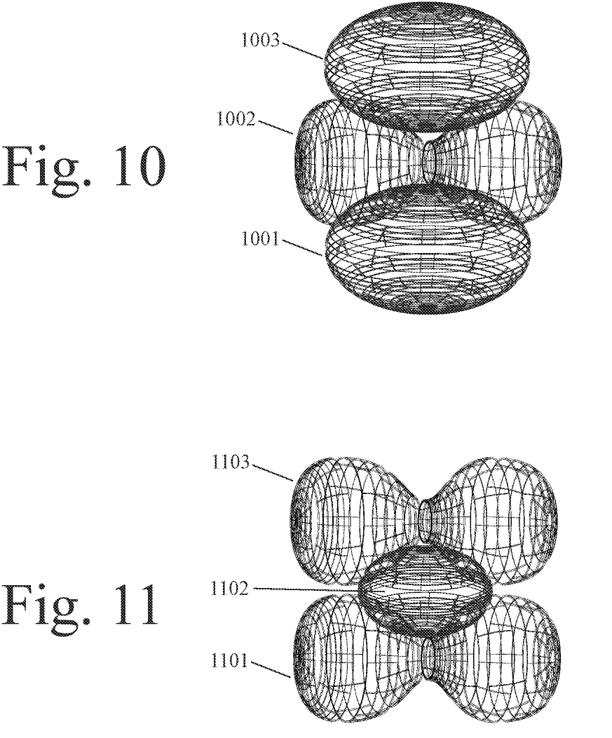

FIG. 10 shows an intrasacular aneurysm occlusion device with a stack of embolic structures comprising: a proximal embolic structure 1001 which is configured to be in an aneurysm sac a first distance from the aneurysm neck; a middle embolic structure 1002 which is configured to be in the aneurysm sac a second distance from the aneurysm neck; and a distal embolic structure 1003 which is configured to be in the aneurysm sac a third distance from the aneurysm neck; wherein the second distance is greater than the first distance; wherein the third distance is greater than the second distance; wherein central axes of the proximal embolic structure, the middle embolic structure, and the distal embolic structure are linearly aligned; wherein the proximal embolic structure and the distal embolic structure have shapes selected from the group consisting of ellipsoid, oblate spheroid, and ovaloid; and wherein the middle embolic structure has a peanut shape. Example variations discussed elsewhere in this disclosure or priority-linked disclosures can also be applied to this example where relevant.

FIG. 11 shows an intrasacular aneurysm occlusion device with a stack of embolic structures comprising: a proximal embolic structure 1101 which is configured to be in an aneurysm sac a first distance from the aneurysm neck; a middle embolic structure 1102 which is configured to be in the aneurysm sac a second distance from the aneurysm neck; and a distal embolic structure 1103 which is configured to be in the aneurysm sac a third distance from the aneurysm neck; wherein the second distance is greater than the first distance; wherein the third distance is greater than the second distance; wherein central axes of the proximal embolic structure, the middle embolic structure, and the distal embolic structure are linearly aligned; wherein the middle embolic structure has a shape selected from the group consisting of ellipsoid, oblate spheroid, and ovaloid; and wherein the proximal embolic structure and the distal embolic structure have peanut shapes. Example variations discussed elsewhere in this disclosure or priority-linked disclosures can also be applied to this example where relevant.

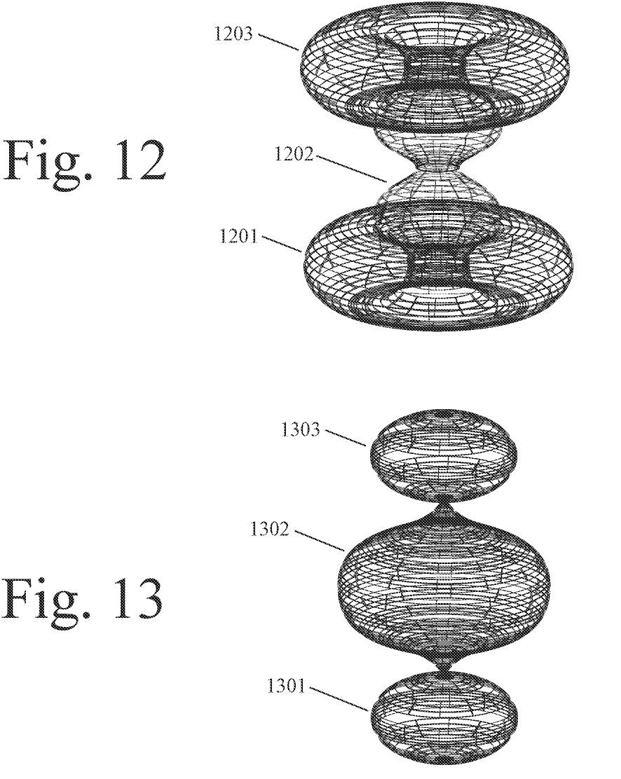

FIG. 12 shows an intrasacular aneurysm occlusion device with a stack of embolic structures comprising: a proximal embolic structure 1201 which is configured to be in an aneurysm sac a first distance from the aneurysm neck; a middle embolic structure 1202 which is configured to be in the aneurysm sac a second distance from the aneurysm neck; and a distal embolic structure 1203 which is configured to be in the aneurysm sac a third distance from the aneurysm neck; wherein the second distance is greater than the first distance; wherein the third distance is greater than the second distance; wherein central axes of the proximal embolic structure, the middle embolic structure, and the distal embolic structure are linearly aligned; wherein the proximal embolic structure and the distal embolic structure have toroidal and/or doughnut shapes; and wherein the middle embolic structure has a (vertical or longitudinally-aligned) peanut shape. Example variations discussed elsewhere in this disclosure or priority-linked disclosures can also be applied to this example where relevant.

FIG. 13 shows an intrasacular aneurysm occlusion device with a stack of embolic structures comprising: a proximal embolic structure 1301 which is configured to be in an aneurysm sac a first distance from the aneurysm neck; a middle embolic structure 1302 which is configured to be in the aneurysm sac a second distance from the aneurysm neck; and a distal embolic structure 1303 which is configured to be in the aneurysm sac a third distance from the aneurysm neck; wherein the second distance is greater than the first distance; wherein the third distance is greater than the second distance; wherein central axes of the proximal embolic structure, the middle embolic structure, and the distal embolic structure are linearly aligned; wherein the proximal embolic structure and the distal embolic structure have shapes selected from the group consisting of ellipsoid, oblate spheroid, and ovaloid; and wherein the middle embolic structure has a tree ornament shape. Alternatively, the proximal, middle, and distal embolic members can all be the same width. Example variations discussed elsewhere in this disclosure or priority-linked disclosures can also be applied to this example where relevant.

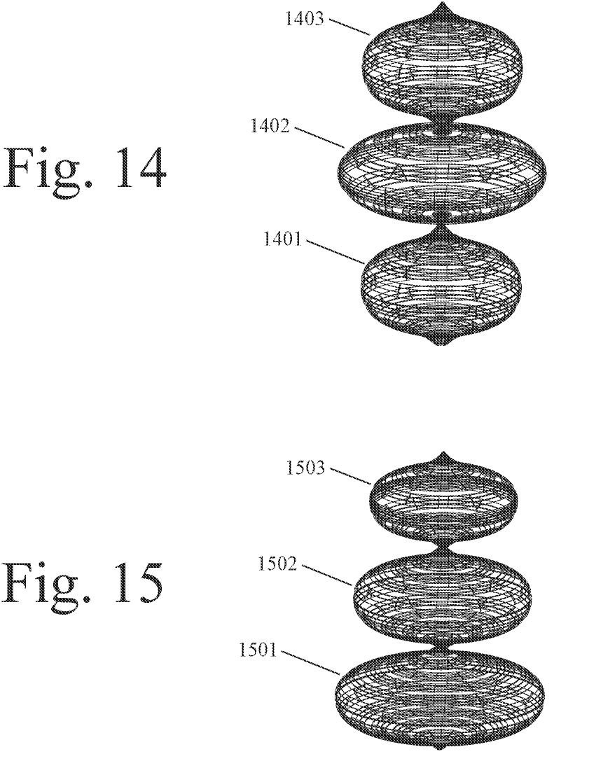

FIG. 14 shows an intrasacular aneurysm occlusion device with a stack of embolic structures comprising: a proximal embolic structure 1401 which is configured to be in an aneurysm sac a first distance from the aneurysm neck; a middle embolic structure 1402 which is configured to be in the aneurysm sac a second distance from the aneurysm neck; and a distal embolic structure 1403 which is configured to be in the aneurysm sac a third distance from the aneurysm neck; wherein the second distance is greater than the first distance; wherein the third distance is greater than the second distance; wherein central axes of the proximal embolic structure, the middle embolic structure, and the distal embolic structure are linearly aligned; wherein the middle embolic structure has a shape selected from the group consisting of ellipsoid, oblate spheroid, and ovaloid; and wherein the proximal embolic structure and the distal embolic structure have tree ornament shapes. Alternatively, the proximal, middle, and distal embolic members can all be the same width. Example variations discussed elsewhere in this disclosure or priority-linked disclosures can also be applied to this example where relevant.

FIG. 15 shows an intrasacular aneurysm occlusion device with a stack of embolic structures comprising: a proximal embolic structure 1501 which is configured to be in an aneurysm sac a first distance from the aneurysm neck; a middle embolic structure 1502 which is configured to be in the aneurysm sac a second distance from the aneurysm neck; and a distal embolic structure 1503 which is configured to be in the aneurysm sac a third distance from the aneurysm neck; wherein the second distance is greater than the first distance; wherein the third distance is greater than the second distance; wherein central axes of the proximal embolic structure, the middle embolic structure, and the distal embolic structure are linearly aligned; wherein the proximal, middle, and distal embolic structures have tree ornament shapes. Alternatively, the proximal, middle, and distal embolic members can all be the same width. Example variations discussed elsewhere in this disclosure or priority-linked disclosures can also be applied to this example where relevant.

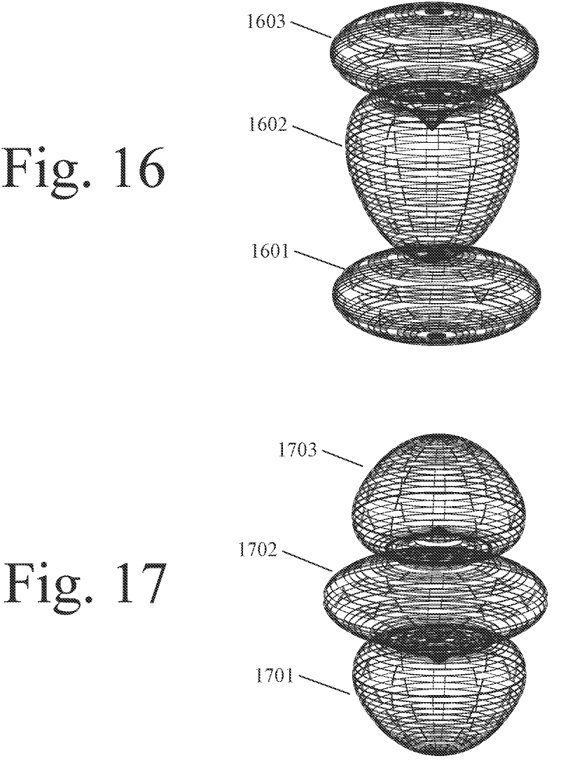

FIG. 16 shows an intrasacular aneurysm occlusion device with a stack of embolic structures comprising: a proximal embolic structure 1601 which is configured to be in an aneurysm sac a first distance from the aneurysm neck; a middle embolic structure 1602 which is configured to be in the aneurysm sac a second distance from the aneurysm neck; and a distal embolic structure 1603 which is configured to be in the aneurysm sac a third distance from the aneurysm neck; wherein the second distance is greater than the first distance; wherein the third distance is greater than the second distance; wherein central axes of the proximal embolic structure, the middle embolic structure, and the distal embolic structure are linearly aligned; wherein the proximal embolic structure and the distal embolic structure have shapes selected from the group consisting of ellipsoid, oblate spheroid, and ovaloid; and wherein the middle embolic structure has a cardioid shape. Alternatively, the proximal, middle, and distal embolic members can all be the same width. Example variations discussed elsewhere in this disclosure or priority-linked disclosures can also be applied to this example where relevant.

FIG. 17 shows an intrasacular aneurysm occlusion device with a stack of embolic structures comprising: a proximal embolic structure 1701 which is configured to be in an aneurysm sac a first distance from the aneurysm neck; a middle embolic structure 1702 which is configured to be in the aneurysm sac a second distance from the aneurysm neck; and a distal embolic structure 1703 which is configured to be in the aneurysm sac a third distance from the aneurysm neck; wherein the second distance is greater than the first distance; wherein the third distance is greater than the second distance; wherein central axes of the proximal embolic structure, the middle embolic structure, and the distal embolic structure are linearly aligned; wherein the middle embolic structure has a shape selected from the group consisting of ellipsoid, oblate spheroid, and ovaloid; and wherein the proximal embolic structure and the distal embolic structure have cardioid shapes. Example variations discussed elsewhere in this disclosure or priority-linked disclosures can also be applied to this example where relevant.

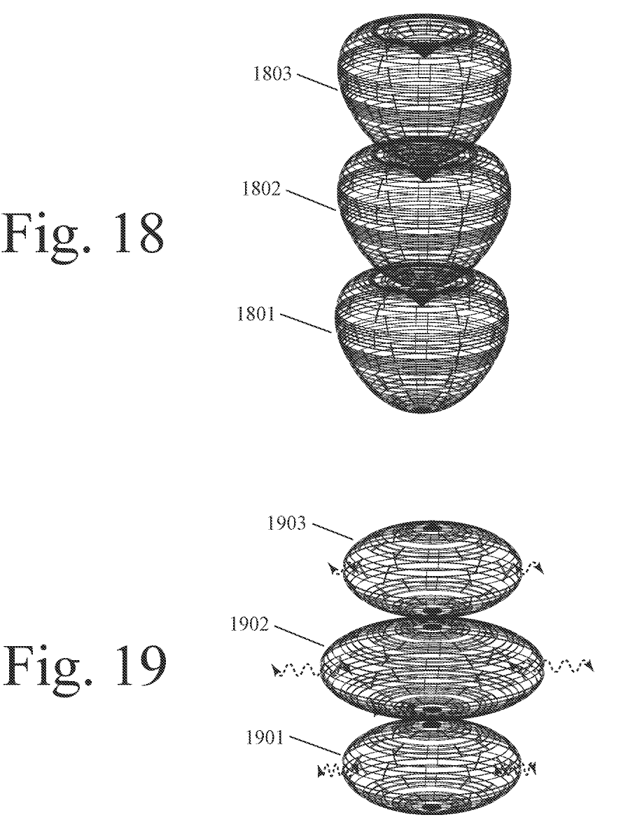

FIG. 18 shows an intrasacular aneurysm occlusion device with a stack of embolic structures comprising: a proximal embolic structure 1801 which is configured to be in an aneurysm sac a first distance from the aneurysm neck; a middle embolic structure 1802 which is configured to be in the aneurysm sac a second distance from the aneurysm neck; and a distal embolic structure 1803 which is configured to be in the aneurysm sac a third distance from the aneurysm neck; wherein the second distance is greater than the first distance; wherein the third distance is greater than the second distance; wherein central axes of the proximal embolic structure, the middle embolic structure, and the distal embolic structure are linearly aligned; wherein the proximal, middle, and distal embolic structures have cardioid shapes. Example variations discussed elsewhere in this disclosure or priority-linked disclosures can also be applied to this example where relevant.

FIG. 19 shows an intrasacular aneurysm occlusion device with a stack of embolic structures comprising: a proximal embolic structure 1901 which is configured to be in an aneurysm sac a first distance from the aneurysm neck; a middle embolic structure 1902 which is configured to be in the aneurysm sac a second distance from the aneurysm neck; and a distal embolic structure 1903 which is configured to be in the aneurysm sac a third distance from the aneurysm neck; wherein the second distance is greater than the first distance; wherein the third distance is greater than the second distance; wherein central axes of the proximal embolic structure, the middle embolic structure, and the distal embolic structure are linearly aligned; wherein the proximal embolic structure, the middle embolic structure, and the distal embolic structure have shapes selected from the group consisting of ellipsoid, oblate spheroid, and ovaloid; wherein the middle embolic structure is between 25% and 250% wider than each of the proximal embolic structure and the distal embolic structure; and wherein the middle embolic structure has a higher level of flexibility, a higher level of elasticity, and/or a lower durometer level than the proximal embolic structure and the distal embolic structure. Alternatively, the proximal, middle, and distal embolic members can all be the same width. Example variations discussed elsewhere in this disclosure or priority-linked disclosures can also be applied to this example where relevant.

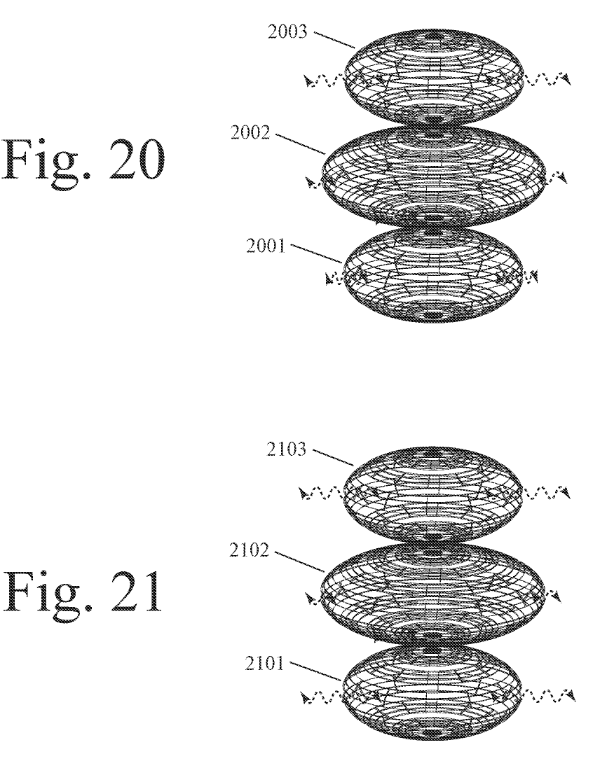

FIG. 20 shows an intrasacular aneurysm occlusion device with a stack of embolic structures comprising: a proximal embolic structure 2001 which is configured to be in an aneurysm sac a first distance from the aneurysm neck; a middle embolic structure 2002 which is configured to be in the aneurysm sac a second distance from the aneurysm neck; and a distal embolic structure 2003 which is configured to be in the aneurysm sac a third distance from the aneurysm neck; wherein the second distance is greater than the first distance; wherein the third distance is greater than the second distance; wherein central axes of the proximal embolic structure, the middle embolic structure, and the distal embolic structure are linearly aligned; wherein the proximal embolic structure, the middle embolic structure, and the distal embolic structure have shapes selected from the group consisting of ellipsoid, oblate spheroid, and ovaloid; wherein the middle embolic structure is between 25% and 250% wider than each of the proximal embolic structure and the distal embolic structure; and wherein the distal embolic structure has a higher level of flexibility, a higher level of elasticity, and/or a lower durometer level than the proximal embolic structure and the middle embolic structure. Alternatively, the proximal, middle, and distal embolic members can all be the same width. Example variations discussed elsewhere in this disclosure or priority-linked disclosures can also be applied to this example where relevant.

FIG. 21 shows an intrasacular aneurysm occlusion device with a stack of embolic structures comprising: a proximal embolic structure 2101 which is configured to be in an aneurysm sac a first distance from the aneurysm neck; a middle embolic structure 2102 which is configured to be in the aneurysm sac a second distance from the aneurysm neck; and a distal embolic structure 2103 which is configured to be in the aneurysm sac a third distance from the aneurysm neck; wherein the second distance is greater than the first distance; wherein the third distance is greater than the second distance; wherein central axes of the proximal embolic structure, the middle embolic structure, and the distal embolic structure are linearly aligned; wherein the proximal embolic structure, the middle embolic structure, and the distal embolic structure have shapes selected from the group consisting of ellipsoid, oblate spheroid, and ovaloid; wherein the middle embolic structure is between 25% and 250% wider than each of the proximal embolic structure and the distal embolic structure; and wherein the proximal embolic structure and the distal embolic structure have a higher level of flexibility, a higher level of elasticity, and/or a lower durometer level than the middle embolic structure. Alternatively, the proximal, middle, and distal embolic members can all be the same width. Example variations discussed elsewhere in this disclosure or priority-linked disclosures can also be applied to this example where relevant.

FIG. 22 shows an intrasacular aneurysm occlusion device with a stack of embolic structures comprising: a proximal embolic structure 2201 which is configured to be in an aneurysm sac a first distance from the aneurysm neck; a middle embolic structure 2202 which is configured to be in the aneurysm sac a second distance from the aneurysm neck; and a distal embolic structure 2203 which is configured to be in the aneurysm sac a third distance from the aneurysm neck; wherein the second distance is greater than the first distance; wherein the third distance is greater than the second distance; wherein central axes of the proximal embolic structure, the middle embolic structure, and the distal embolic structure are linearly aligned; wherein the proximal embolic structure, the middle embolic structure, and the distal embolic structure have shapes selected from the group consisting of ellipsoid, oblate spheroid, and ovaloid; wherein the middle embolic structure is between 25% and 250% wider than each of the proximal embolic structure and the distal embolic structure; and wherein embolic material 2204 is inserted into the interior of the proximal embolic structure after the device has been inserted into the aneurysm sac. Alternatively, the proximal, middle, and distal embolic members can all be the same width. Example variations discussed elsewhere in this disclosure or priority-linked disclosures can also be applied to this example where relevant.

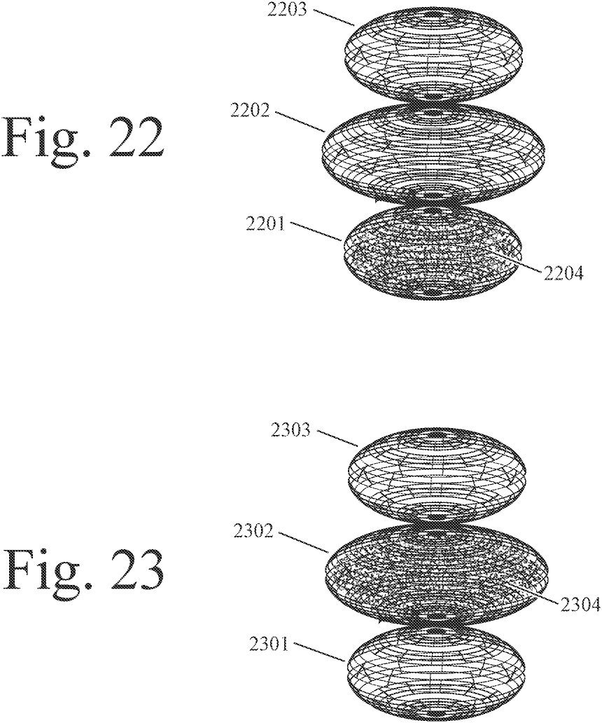

FIG. 23 shows an intrasacular aneurysm occlusion device with a stack of embolic structures comprising: a proximal embolic structure 2301 which is configured to be in an aneurysm sac a first distance from the aneurysm neck; a middle embolic structure 2302 which is configured to be in the aneurysm sac a second distance from the aneurysm neck; and a distal embolic structure 2303 which is configured to be in the aneurysm sac a third distance from the aneurysm neck; wherein the second distance is greater than the first distance; wherein the third distance is greater than the second distance; wherein central axes of the proximal embolic structure, the middle embolic structure, and the distal embolic structure are linearly aligned; wherein the proximal embolic structure, the middle embolic structure, and the distal embolic structure have shapes selected from the group consisting of ellipsoid, oblate spheroid, and ovaloid; wherein the middle embolic structure is between 25% and 250% wider than each of the proximal embolic structure and the distal embolic structure; and wherein embolic material 2304 is inserted into the interior of the middle embolic structure after the device has been inserted into the aneurysm sac. Alternatively, the proximal, middle, and distal embolic members can all be the same width. Example variations discussed elsewhere in this disclosure or priority-linked disclosures can also be applied to this example where relevant.

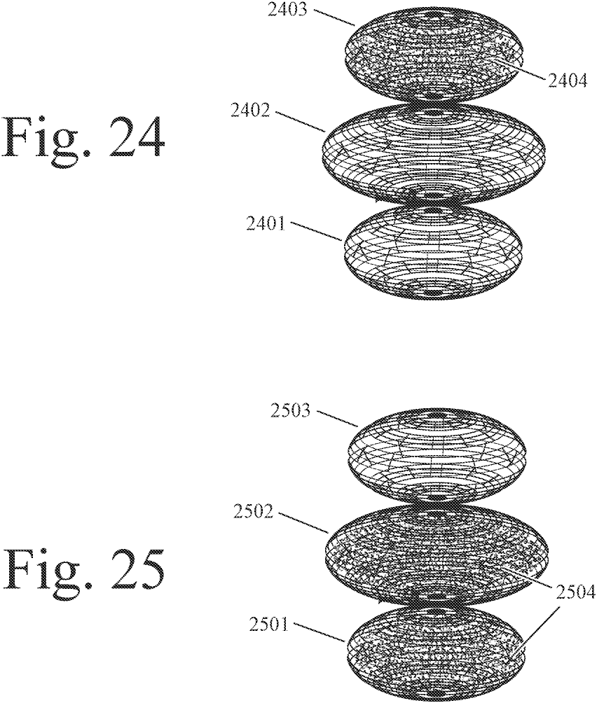

FIG. 24 shows an intrasacular aneurysm occlusion device with a stack of embolic structures comprising: a proximal embolic structure 2401 which is configured to be in an aneurysm sac a first distance from the aneurysm neck; a middle embolic structure 2402 which is configured to be in the aneurysm sac a second distance from the aneurysm neck; and a distal embolic structure 2403 which is configured to be in the aneurysm sac a third distance from the aneurysm neck; wherein the second distance is greater than the first distance; wherein the third distance is greater than the second distance; wherein central axes of the proximal embolic structure, the middle embolic structure, and the distal embolic structure are linearly aligned; wherein the proximal embolic structure, the middle embolic structure, and the distal embolic structure have shapes selected from the group consisting of ellipsoid, oblate spheroid, and ovaloid; wherein the middle embolic structure is between 25% and 250% wider than each of the proximal embolic structure and the distal embolic structure; and wherein embolic material 2404 is inserted into the interior of the distal embolic structure after the device has been inserted into the aneurysm sac. Alternatively, the proximal, middle, and distal embolic members can all be the same width. Example variations discussed elsewhere in this disclosure or priority-linked disclosures can also be applied to this example where relevant.

FIG. 25 shows an intrasacular aneurysm occlusion device with a stack of embolic structures comprising: a proximal embolic structure 2501 which is configured to be in an aneurysm sac a first distance from the aneurysm neck; a middle embolic structure 2502 which is configured to be in the aneurysm sac a second distance from the aneurysm neck; and a distal embolic structure 2503 which is configured to be in the aneurysm sac a third distance from the aneurysm neck; wherein the second distance is greater than the first distance; wherein the third distance is greater than the second distance; wherein central axes of the proximal embolic structure, the middle embolic structure, and the distal embolic structure are linearly aligned; wherein the proximal embolic structure, the middle embolic structure, and the distal embolic structure have shapes selected from the group consisting of ellipsoid, oblate spheroid, and ovaloid; wherein the middle embolic structure is between 25% and 250% wider than each of the proximal embolic structure and the distal embolic structure; and wherein embolic material 2504 is inserted into the interiors of the proximal embolic structure and the middle embolic structure after the device has been inserted into the aneurysm sac. Alternatively, the proximal, middle, and distal embolic members can all be the same width. Example variations discussed elsewhere in this disclosure or priority-linked disclosures can also be applied to this example where relevant.

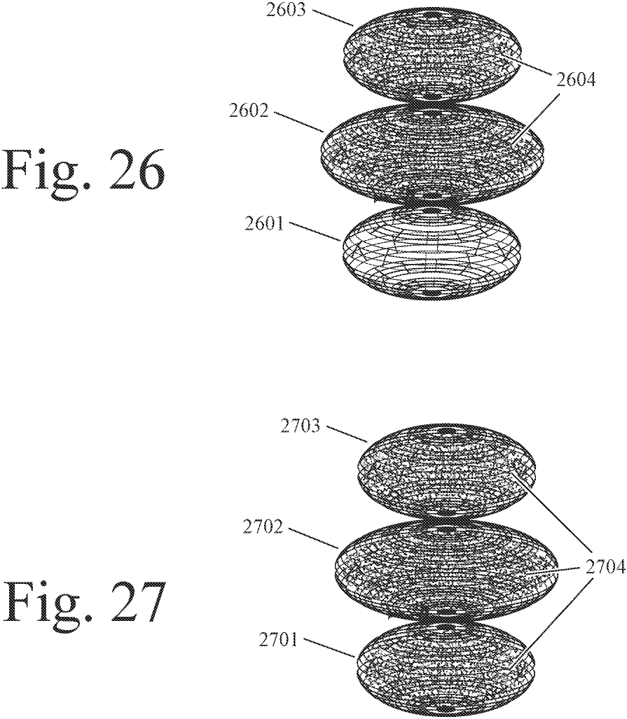

FIG. 26 shows an intrasacular aneurysm occlusion device with a stack of embolic structures comprising: a proximal embolic structure 2601 which is configured to be in an aneurysm sac a first distance from the aneurysm neck; a middle embolic structure 2602 which is configured to be in the aneurysm sac a second distance from the aneurysm neck; and a distal embolic structure 2603 which is configured to be in the aneurysm sac a third distance from the aneurysm neck; wherein the second distance is greater than the first distance; wherein the third distance is greater than the second distance; wherein central axes of the proximal embolic structure, the middle embolic structure, and the distal embolic structure are linearly aligned; wherein the proximal embolic structure, the middle embolic structure, and the distal embolic structure have shapes selected from the group consisting of ellipsoid, oblate spheroid, and ovaloid; wherein the middle embolic structure is between 25% and 250% wider than each of the proximal embolic structure and the distal embolic structure; and wherein embolic material 2604 is inserted into the interiors of the middle embolic structure and the distal embolic structure after the device has been inserted into the aneurysm sac. Alternatively, the proximal, middle, and distal embolic members can all be the same width. Example variations discussed elsewhere in this disclosure or priority-linked disclosures can also be applied to this example where relevant.

FIG. 27 shows an intrasacular aneurysm occlusion device with a stack of embolic structures comprising: a proximal embolic structure 2701 which is configured to be in an aneurysm sac a first distance from the aneurysm neck; a middle embolic structure 2702 which is configured to be in the aneurysm sac a second distance from the aneurysm neck; and a distal embolic structure 2703 which is configured to be in the aneurysm sac a third distance from the aneurysm neck; wherein the second distance is greater than the first distance; wherein the third distance is greater than the second distance; wherein central axes of the proximal embolic structure, the middle embolic structure, and the distal embolic structure are linearly aligned; wherein the proximal embolic structure, the middle embolic structure, and the distal embolic structure have shapes selected from the group consisting of ellipsoid, oblate spheroid, and ovaloid; wherein the middle embolic structure is between 25% and 250% wider than each of the proximal embolic structure and the distal embolic structure; and wherein embolic material 2704 is inserted into the interiors of the proximal embolic structure, the middle embolic structure, and the distal embolic structure after the device has been inserted into the aneurysm sac. Alternatively, the proximal, middle, and distal embolic members can all be the same width. Example variations discussed elsewhere in this disclosure or priority-linked disclosures can also be applied to this example where relevant.

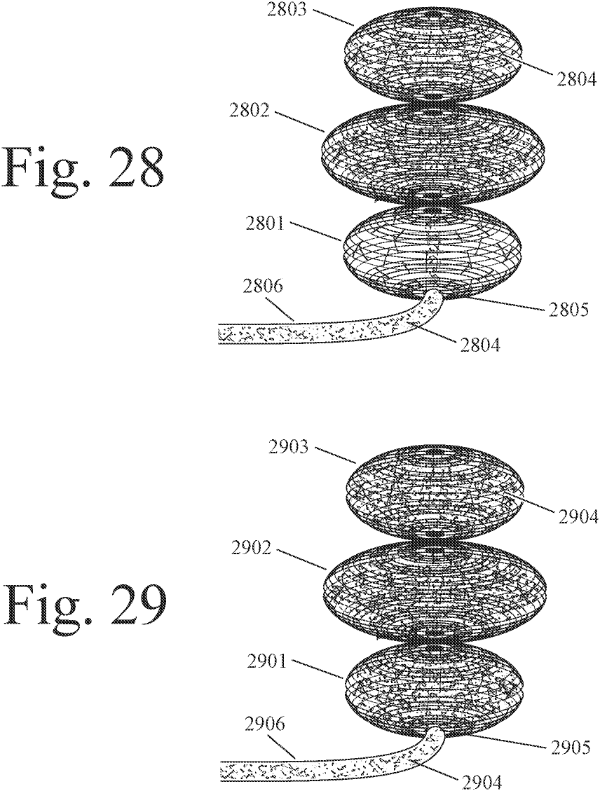

FIG. 28 shows an intrasacular aneurysm occlusion device with a stack of embolic structures comprising: a proximal embolic structure 2801 which is configured to be in an aneurysm sac a first distance from the aneurysm neck; a middle embolic structure 2802 which is configured to be in the aneurysm sac a second distance from the aneurysm neck; a distal embolic structure 2803 which is configured to be in the aneurysm sac a third distance from the aneurysm neck; wherein the second distance is greater than the first distance; wherein the third distance is greater than the second distance; wherein central axes of the proximal embolic structure, the middle embolic structure, and the distal embolic structure are linearly aligned; wherein the proximal embolic structure, the middle embolic structure, and the distal embolic structure have shapes selected from the group consisting of ellipsoid, oblate spheroid, and ovaloid; wherein the middle embolic structure is between 25% and 250% wider than each of the proximal embolic structure and the distal embolic structure; an opening 2805 through the proximal embolic structure; a catheter 2806; and embolic members and/or material 2804, wherein the embolic members and/or material is delivered through the catheter and the opening into the interiors of the middle embolic structure and the distal embolic structure after the device has been inserted into the aneurysm sac.

In an example, a stack of embolic structures can be a connected longitudinal series of linearly-aligned embolic structures. In an example, an embolic structure can be a mesh, net, braid, and/or stent. In an example, an embolic structure can have a longitudinally-expanded and radially-compressed configuration as it is delivered through a catheter to an aneurysm sac. In an example, an embolic structure can shrink longitudinally and expand radially after it leaves the catheter in the aneurysm sac. In an example, a stack of embolic structures can be a longitudinal series of connected embolic structures.

In an example, embolic structures in a stack can all have the same shape. In an example, the embolic structures can have different shapes. In an example, the shape of a proximal, middle, and/or distal embolic structure can be selected from the group consisting of: spherical shape, ellipsoidal shape, oblate spheroid shape, barrel shape, canister shape, racetrack ovaloid shape, disk shape, apple shape, pumpkin shape, toroidal shape, doughnut shape, tree ornament (3D revolved sine-wave), egg shape, pear shape, tear-drop shape, cardioid shape, bullet shape, hemispherical shape, bowl shape, half-canister shape, funnel shape, jellyfish shape, 3D revolved horseshoe shape, jug or bottle shape, mushroom shape, hour-glass shape, hyperboloidal shape, peanut shape, dumbbell shape, reflected mushroom shape, Saturn shape, paper lantern shape, and geodesic shape. In an example, a one or more embolic structures in a stack can have ellipsoidal, oblate spheroid, or ring shape and one or more of the other embolic structures in the stack can have a toroidal, doughnut, or peanut shape. In an example, the shape of one or more embolic structures in a stack of embolic structures can be adjusted, controlled, and/or varied in real time by a device operator in order to best match the contours of a specific aneurysm sac.

In an example, embolic structures in a stack can all be the same size. In an example, embolic structures in a stack can all be the same width and/or diameter. In an example, a middle embolic structure can be wider (e.g. 25% to 200% wider) than each of a proximal embolic structure and a distal embolic structure. This design can help to keep the structure in the aneurysm sac if the structure engages the walls of the aneurysm sac at its widest section (e.g. if the middle section of the sac is widest). This can be optimal for berry aneurysms. In an example, a proximal embolic structure can be wider (e.g. 25% to 200% wider) than each of a middle embolic structure and a distal embolic structure. This design can help to maximize coverage of the aneurysm neck to minimize flow of blood into the aneurysm sac. This design can also be optimal for fusiform aneurysms. In an example, proximal and distal embolic structures can each be wider (e.g. 25% to 200% wider) than a middle embolic structure. This design can help the device to bend laterally, which can be help to occlude asymmetric and/or irregularly-shaped aneurysms. In an example, the size of one or more embolic structures in a stack of embolic structures can be adjusted, controlled, and/or varied in real time by a device operator in order to best match the contours of a specific aneurysm sac.

In an example, embolic structures in a stack can all have the same level of flexibility, elasticity, and/or durometer. In an example, a middle embolic structure can have greater flexibility, greater elasticity, and/or lower durometer than each of a proximal embolic structure and a distal embolic structure. In an example, a distal embolic structure can have greater flexibility, greater elasticity, and/or lower durometer than each of a proximal embolic structure and a middle embolic structure. In an example, proximal and distal embolic structures can have greater flexibility, greater elasticity, and/or lower durometer than a middle embolic structure. In an example, embolic structures in a stack can all have the same porosity level. In an example, a middle embolic structure can have greater porosity than each of a proximal embolic structure and a distal embolic structure. In an example, a distal embolic structure can have greater porosity than each of a proximal embolic structure and a middle embolic structure.

In an example, the interiors of one or more embolic structures in a stack can be filled with embolic members and/or material after the one or more embolic structures have been inserted into an aneurysm sac. In an example, embolic members and/or material can be selected from the group consisting of: embolic coils, hydrogels, microsponges, beads, ribbons, string-of-pearls strands, and congealing material. In an example, an embolic structure can further expand when it is filled with embolic members and/or material so that better conforms to the walls of an irregularly-shaped aneurysm sac and reduces the risk of recanalization.

In an example, there can be an opening in an embolic structure through which embolic members and/or material is inserted into the interior of the embolic structure. In an example, the opening can be through the central axis of an embolic structure. In an example, the opening can include a valve which can be closed remotely by the operator of the device. In an example, all three embolic structures in a stack can be filled with embolic members and/or material. In an example, only one or two of three embolic structures in a stack are filled with embolic members and/or material.

In an example, a valve in an opening can passively open when an embolic member is pushed through it and can passively close after the member passes through or when a portion of the member is detached. In an example, such a valve allows an embolic member to be inserted into an aneurysm, but the valve closes to reduce blood flow into the aneurysm after the embolic member has passed through the valve. In an example, an active valve can be remotely opened and/or closed by the operator of the device. In an example, an active valve can be remotely opened and/or closed by an operator by the application of electromagnetic energy. In an example, an active valve can be remotely opened and/or closed by an operator by pulling a filament. In an example, an active valve can be remotely opened and/or closed by an operator by pushing, pulling, or rotating a wire. In an example, an active valve can be remotely opened and/or closed by an operator by cutting, pulling, or pushing a flap or plug.

In an example, proximal, middle, and distal embolic structures can be created separately and then connected to each other. In an example, proximal, middle, and distal embolic structures can be connected by a longitudinal wire, cord, string, strand, or coil. In an example, the longitudinal axes of proximal, middle, and distal embolic structures can be connected by a longitudinal wire, cord, string, strand, or coil. Alternatively, proximal, middle, and distal embolic structures can be formed from a common and/or continuous component. In an example, proximal, middle, and distal embolic structures can be formed by radially-constraining segments of a tubular mesh. In an example, proximal, middle, and distal embolic structures can be lobes of a radially-constrained tubular mesh. In an example, proximal, middle, and distal embolic structures can be formed by radially-constraining a tubular mesh with bands and/or rings. In an example, proximal, middle, and distal embolic structures can be formed by radially-constraining a tubular mesh at multiple locations. In an example, proximal, middle, and distal embolic structures can be formed by radially-constraining and inverting a tubular mesh. In an example, proximal, middle, and distal embolic structures can be formed by radially-constraining and everting a tubular mesh. Example variations discussed elsewhere in this disclosure or priority-linked disclosures can also be applied to this example where relevant.

FIG. 29 shows an intrasacular aneurysm occlusion device with a stack of embolic structures comprising: a proximal embolic structure 2901 which is configured to be in an aneurysm sac a first distance from the aneurysm neck; a middle embolic structure 2902 which is configured to be in the aneurysm sac a second distance from the aneurysm neck; a distal embolic structure 2903 which is configured to be in the aneurysm sac a third distance from the aneurysm neck; wherein the second distance is greater than the first distance; wherein the third distance is greater than the second distance; wherein central axes of the proximal embolic structure, the middle embolic structure, and the distal embolic structure are linearly aligned; wherein the proximal embolic structure, the middle embolic structure, and the distal embolic structure have shapes selected from the group consisting of ellipsoid, oblate spheroid, and ovaloid; wherein the middle embolic structure is between 25% and 250% wider than each of the proximal embolic structure and the distal embolic structure; an opening 2905 in the proximal embolic structure; a catheter 2906; and embolic members and/or material 2904, wherein the embolic members and/or material is delivered through the catheter and the opening into the interiors of the proximal embolic structure, the middle embolic structure, and the distal embolic structure after the device has been inserted into the aneurysm sac. Example variations discussed elsewhere in this disclosure or priority-linked disclosures can also be applied to this example where relevant.

In an example, a stack of embolic structures can be a connected longitudinal series of centrally-aligned embolic structures. In an example, an embolic structure can be a mesh, net, braid, and/or stent. In an example, an embolic structure can have a longitudinally-expanded and radially-compressed configuration as it is delivered through a catheter to an aneurysm sac. In an example, an embolic structure can shrink longitudinally and expand radially after it leaves the catheter in the aneurysm sac. In an example, a stack of embolic structures can be a longitudinal series of connected embolic structures.

In an example, embolic structures in a stack can all have the same shape. In an example, the embolic structures can have different shapes. In an example, the shape of a proximal, middle, and/or distal embolic structure can be selected from the group consisting of: spherical shape, ellipsoidal shape, oblate spheroid shape, barrel shape, canister shape, racetrack ovaloid shape, disk shape, apple shape, pumpkin shape, toroidal shape, doughnut shape, tree ornament (3D revolved sine-wave), egg shape, pear shape, tear-drop shape, cardioid shape, bullet shape, hemispherical shape, bowl shape, half-canister shape, funnel shape, jellyfish shape, 3D revolved horseshoe shape, jug or bottle shape, mushroom shape, hour-glass shape, hyperboloidal shape, peanut shape, dumbbell shape, reflected mushroom shape, Saturn shape, paper lantern shape, and geodesic shape. In an example, a one or more embolic structures in a stack can have ellipsoidal, oblate spheroid, or ring shape and one or more of the other embolic structures in the stack can have a toroidal, doughnut, or peanut shape. In an example, the shape of one or more embolic structures in a stack of embolic structures can be adjusted, controlled, and/or varied in real time by a device operator in order to best match the contours of a specific aneurysm sac.

In an example, embolic structures in a stack can all be the same size. In an example, embolic structures in a stack can all be the same width and/or diameter. In an example, a middle embolic structure can be wider (e.g. 25% to 200% wider) than each of a proximal embolic structure and a distal embolic structure. This design can help to keep the structure in the aneurysm sac if the structure engages the walls of the aneurysm sac at its widest section (e.g. if the middle section of the sac is widest). This can be optimal for berry aneurysms. In an example, a proximal embolic structure can be wider (e.g. 25% to 200% wider) than each of a middle embolic structure and a distal embolic structure. This design can help to maximize coverage of the aneurysm neck to minimize flow of blood into the aneurysm sac. This design can also be optimal for fusiform aneurysms. In an example, proximal and distal embolic structures can each be wider (e.g. 25% to 200% wider) than a middle embolic structure. This design can help the device to bend laterally, which can be help to occlude asymmetric and/or irregularly-shaped aneurysms. In an example, the size of one or more embolic structures in a stack of embolic structures can be adjusted, controlled, and/or varied in real time by a device operator in order to best match the contours of a specific aneurysm sac.

In an example, embolic structures in a stack can all have the same level of flexibility, elasticity, and/or durometer. In an example, a middle embolic structure can have greater flexibility, greater elasticity, and/or lower durometer than each of a proximal embolic structure and a distal embolic structure. In an example, a distal embolic structure can have greater flexibility, greater elasticity, and/or lower durometer than each of a proximal embolic structure and a middle embolic structure. In an example, proximal and distal embolic structures can have greater flexibility, greater elasticity, and/or lower durometer than a middle embolic structure. In an example, embolic structures in a stack can all have the same porosity level. In an example, a middle embolic structure can have greater porosity than each of a proximal embolic structure and a distal embolic structure. In an example, a distal embolic structure can have greater porosity than each of a proximal embolic structure and a middle embolic structure.

In an example, the interiors of one or more embolic structures in a stack can be filled with embolic members and/or material after the one or more embolic structures have been inserted into an aneurysm sac. In an example, embolic members and/or material can be selected from the group consisting of: embolic coils, hydrogels, microsponges, beads, ribbons, string-of-pearls strands, and congealing material. In an example, an embolic structure can further expand when it is filled with embolic members and/or material so that better conforms to the walls of an irregularly-shaped aneurysm sac and reduces the risk of recanalization.

In an example, there can be an opening in an embolic structure through which embolic members and/or material is inserted into the interior of the embolic structure. In an example, the opening can be through the central axis of an embolic structure. In an example, the opening can include a valve which can be closed remotely by the operator of the device. In an example, all three embolic structures in a stack can be filled with embolic members and/or material. In an example, only one or two of three embolic structures in a stack are filled with embolic members and/or material.

In an example, a valve in an opening can passively open when an embolic member is pushed through it and can passively close after the member passes through or when a portion of the member is detached. In an example, such a valve allows an embolic member to be inserted into an aneurysm, but the valve closes to reduce blood flow into the aneurysm after the embolic member has passed through the valve. In an example, an active valve can be remotely opened and/or closed by the operator of the device. In an example, an active valve can be remotely opened and/or closed by an operator by the application of electromagnetic energy. In an example, an active valve can be remotely opened and/or closed by an operator by pulling a filament. In an example, an active valve can be remotely opened and/or closed by an operator by pushing, pulling, or rotating a wire. In an example, an active valve can be remotely opened and/or closed by an operator by cutting, pulling, or pushing a flap or plug.

In an example, proximal, middle, and distal embolic structures can be created separately and then connected to each other. In an example, proximal, middle, and distal embolic structures can be connected by a longitudinal wire, cord, string, strand, or coil. In an example, the longitudinal axes of proximal, middle, and distal embolic structures can be connected by a longitudinal wire, cord, string, strand, or coil. Alternatively, proximal, middle, and distal embolic structures can be formed from a common and/or continuous component. In an example, proximal, middle, and distal embolic structures can be formed by radially-constraining segments of a tubular mesh. In an example, proximal, middle, and distal embolic structures can be lobes of a radially-constrained tubular mesh. In an example, proximal, middle, and distal embolic structures can be formed by radially-constraining a tubular mesh with bands and/or rings. In an example, proximal, middle, and distal embolic structures can be formed by radially-constraining a tubular mesh at multiple locations. In an example, proximal, middle, and distal embolic structures can be formed by radially-constraining and inverting a tubular mesh. In an example, proximal, middle, and distal embolic structures can be formed by radially-constraining and everting a tubular mesh. Example variations discussed elsewhere in this disclosure or priority-linked disclosures can also be applied to this example where relevant.

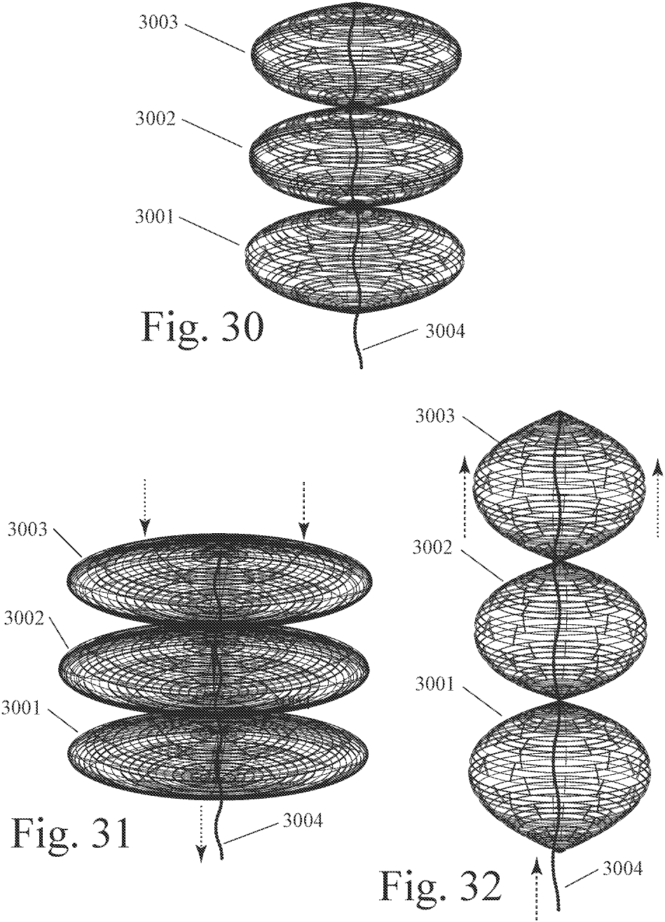

FIGS. 30 through 32 shows three configurations, at three different times, of an intrasacular aneurysm occlusion device with a stack of embolic structures comprising: a proximal embolic structure 3001 which is configured to be deployed in an aneurysm sac at a first distance from the aneurysm neck; a middle embolic structure 3002 which is configured to be deployed in the aneurysm sac at a second distance from the aneurysm neck; and a distal embolic structure 3003 which is configured to be deployed in the aneurysm sac at a third distance from the aneurysm neck; wherein the second distance is greater than the first distance; wherein the third distance is greater than the second distance; and wherein the proximal embolic structure, the middle embolic structure, and the distal embolic structure combine to form a stack with a shape which is a 3D revolution of three single phases (or one and half full phases) of a sine wave around the central longitudinal axis of the sine wave.

In an example, the amplitude and frequency of the 3D revolution formed by the combination of these three embolic structures can be controlled by the operator of the device by moving a wire 3004 which is attached to the structures. FIG. 30 shows this device in a first configuration, wherein the stack of three embolic structures has the shape of a 3D revolution a sine wave with a first amplitude and a first frequency.

FIG. 31 shows this device in a second configuration, wherein the stack of three embolic structures has the shape of a 3D revolution a sine wave with a second amplitude and a second frequency, wherein the second amplitude is greater than the first amplitude, and wherein the second frequency is greater than the first frequency. The stack has been radially-expanded and longitudinally-contracted from the first configuration to the second configuration. In an example, the stack can be moved from the first configuration to the second configuration when the device operator pulls a wire attached to some of the structures and/or pushes some of the structures relative to the wire.

FIG. 32 shows this device in a third configuration, wherein the stack of three embolic structures has the shape of a 3D revolution a sine wave with a third amplitude and a third frequency, wherein the third amplitude is less than the first amplitude, and wherein the third frequency is less than the first frequency. The stack has been radially-contracted and longitudinally-expanded from the first configuration to the third configuration. In an example, the stack can be moved from the first configuration to the third configuration when the device operator pushes a wire attached to some of the structures and/or pulls some of the structures relative to the wire. Example variations discussed elsewhere in this disclosure or priority-linked disclosures can also be applied to this example where relevant.

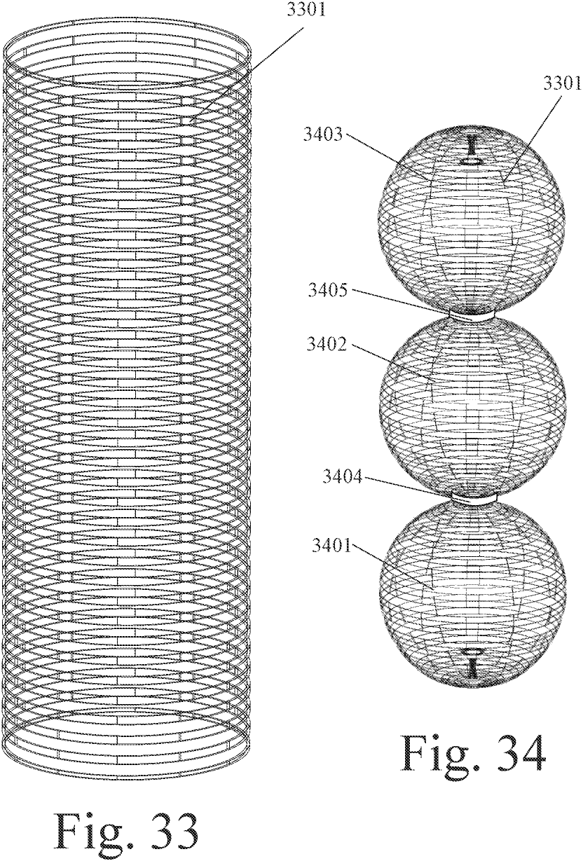

FIGS. 33 through 36 show views at four different times of an example of an intrasacular aneurysm occlusion device with a stack of centrally-aligned embolic structures 3401, 3402, and 3403 which are configured to be inserted and radially-expanded in an aneurysm sac. In this example, embolic structures in the stack of embolic structures are lobes, bulges, and/or segments of a single continuous component 3301. In this example, the continuous component is a tubular mesh (e.g. tubular mesh, braid, and/or stent). In this example, embolic structures in the stack of embolic structures are created by radially-constraining a tubular mesh at multiple locations along its longitudinal axis. In this example, multiple locations along the longitudinal axis of the tubular mesh are radially-constrained by bands, rings, and/or cylinders 3304 and 3305 around the tubular mesh.

In this example, the proximal and distal ends of the tubular mesh are also radially-constrained. In an example, the proximal and distal ends can be radially-constrained by being tied, glued, melted, or soldered. In an example, the proximal and distal ends can be radially-constrained using bands, rings, and/or cylinders, as was the case with radial constraint of mid-sections of the tube. In this example, proximal and distal ends of the tubular mesh are also inverted into the interior of the tubular mesh. In this example, there are three embolic structures in the stack: a proximal embolic structure 3401; a middle embolic structure 3402; and a distal embolic structure 3403. In an example, there may only be two embolic structures: a proximal embolic structure; and a distal embolic structure.

FIG. 33 shows this device at a first time, in a first configuration, before the tubular mesh has been radially-constrained to form a stack of embolic structures. In this first configuration, the device (e.g. the tubular mesh) has a generally-cylindrical shape.

FIG. 34 shows this device at a second time, in a second configuration, after the tubular mesh has been radially-constrained in multiple locations to form a stack of embolic structures, but before the stack has been elongated for insertion into a catheter for delivery through a patient’s vasculature to an aneurysm sac. In this second configuration, the stack has a 3D shape with an undulating, scalloped, and/or sinusoidal longitudinal perimeter. In this example in this second configuration, the embolic structures are generally spherical. In this example in this second configuration, the stack can be described as a series, sequence, and/or chain of connected spherical embolic structures.

FIG. 35 shows this device at a third time, in a third configuration, after the stack of embolic structures has been longitudinally-elongated and radially-compressed for insertion into a catheter and delivery through the catheter to an aneurysm sac. In this third configuration, the stack still has a 3D shape with an undulating, scalloped, and/or sinusoidal longitudinal perimeter, but the width of the stack is narrower and the length of the stack is longer than in the second configuration. In this example in the third configuration, the embolic structures are generally ellipsoidal and the stack can be described as a series, sequence, and/or chain of connected ellipsoidal embolic structures whose longest axes are substantially linearly-aligned with each other.

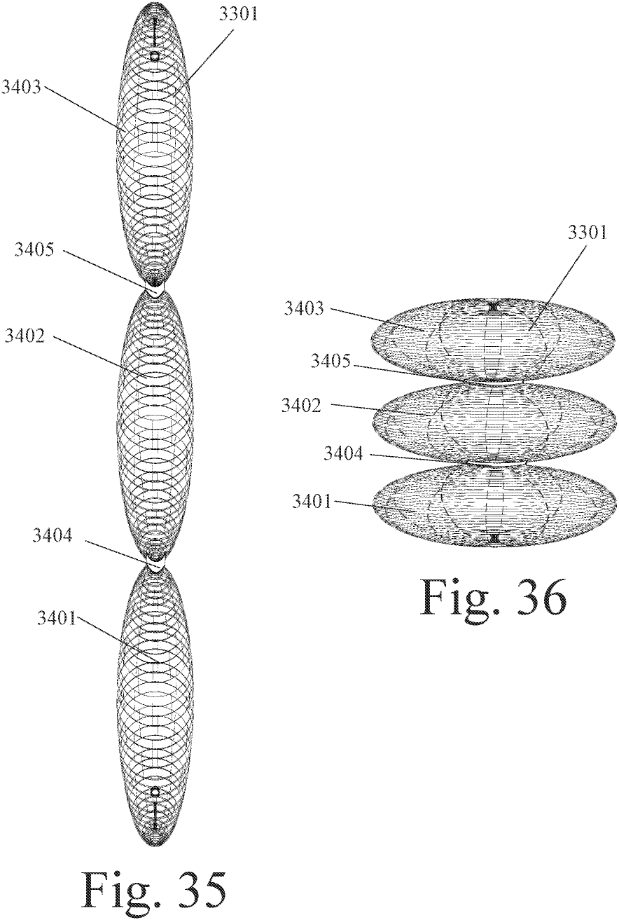

FIG. 36 shows this device at a fourth time, in a fourth configuration, after the stack of embolic structures has exited the catheter, radially-expanded (e.g. self-expanded in a radial direction), and longitudinally-shortened (e.g. self-contracted in a longitudinal direction) inside an aneurysm sac. The radial diameter of the stack is now wider and the length of the stack is now shorter than in the third configuration. In this example, radial expansion and longitudinal compression occurs to such an extent that the orientations of the embolic structures’ shortest and longest axes flip. In the third configuration, the longest axes of the embolic members were generally linearly aligned, but now in this fourth configuration, the radial axes become the longest axes of the embolic members and these axes are generally parallel to each other. In this example in the fourth configuration, the stack can be described as a series, sequence, and/or chain of ellipsoidal embolic structures whose longest axes are substantially parallel to each other. Although not shown in this figure, in this example in this fourth configuration, the longest axes of the ellipsoidal embolic structures can also be substantially parallel to the plane of aneurysm neck.

Each of three embolic structures in this intrasacular stack of embolic structures can serve a purpose. The proximal embolic structure can serve as a neck bridge. The proximal embolic structure covers the neck of the aneurysm from inside the aneurysm sac and prevents blood flow from the parent vessel of the aneurysm into the aneurysm sac. The middle embolic structure serves as an anchor for the device. The middle embolic structure can span the largest diameter of the aneurysm sac and prevent the proximal embolic structure from slipping outward into the parent vessel. The distal embolic structure can help to seal the neck bridge. The distal embolic structure can contact the dome of the aneurysm sac and transmit pressure from this contact with the dome to the proximal embolic structure, pressing the proximal embolic structure against the inside of the aneurysm neck and preventing it from slipping inward into the main body of the aneurysm sac.

In this example, proximal, middle, and distal embolic structures are lobes, bulges, undulations, or segments which are created from a single continuous structure. In this example, proximal, middle, and distal embolic structures are formed by radially-constraining segments of a tubular mesh at multiple locations. In this example, proximal, middle, and distal embolic structures in a stack of embolic structures can have longitudinally-expanded and radially-compressed configurations as they are delivered through a catheter to an aneurysm sac. In this example, these embolic structures shrink longitudinally and expand radially after they exit a catheter in an aneurysm sac. In an example, embolic structures in a stack of embolic structures can be inserted and expanded sequentially within an aneurysm sac, wherein one embolic structure is inserted and expanded before the next embolic structure is inserted into the aneurysm sac. In an example, a plurality of embolic structures in a stack of embolic structures can be inserted and expanded within an aneurysm sac at the same time.

In an example, proximal, middle, and distal embolic structures in a stack of embolic structures can self-expand radially after they exit a catheter in an aneurysm sac. Alternatively, the radial expansion of an embolic structure can be controlled by an operator of the device. In an example, a longitudinal wire, cord, string, strand, tube, catheter, or coil can pass through the centers of proximal, middle, and distal embolic structures. In an example, a device operator can control the amount, direction, and/or timing of radial expansion of an embolic structure by a mechanism selected from the group consisting of: pulling a wire or string; applying electromagnetic energy; and rotating a wire or catheter. In an example, a device operator can selectively control the amount, direction, and/or timing of radial expansion of each embolic structure by a mechanism selected from the group consisting of: pulling a wire or string; applying electromagnetic energy; and rotating a wire or catheter. This can enable the device operator to customize the expanded shape of the stack to conform to the shape and size of an irregularly-shape aneurysm sac.

In an example, the shape of one or more embolic structures in a stack of embolic structures can be adjusted, controlled, and/or varied in real time by a device operator in order to best match the contours of a specific aneurysm sac. In an example, the shape of one or more embolic structures in a stack of embolic structures can be adjusted, controlled, and/or varied in real time by a device operator by a mechanism selected from the group consisting of: pulling a wire, cord, or string; applying electromagnetic energy; and rotating a wire or catheter. In an example, a device operator can control the post-expansion diameter of a proximal, middle, or distal embolic member by one or more of these mechanisms.

In an example, embolic structures in a stack of embolic structures can all be the same size. In an example, embolic structures in a stack can all have the same width and/or diameter. In an example, proximal embolic structures in an intrasacular stack of structures can be larger (e.g. wider and/or longer) than distal embolic structures in the stack. In an example, distal embolic structures in an intrasacular stack of structures can be larger (e.g. wider and/or longer) than proximal embolic structures in the stack. In an example, in a series of embolic structures which are sequentially inserted into an aneurysm sac, embolic structures which are inserted first can be smaller (e.g. narrower) and structures which are inserted later can be larger (e.g. wider). In an example, in a series of embolic structures which are sequentially inserted into an aneurysm sac, embolic structures which are inserted first can be smaller (e.g. narrower), structures which are inserted next can be larger (e.g. wider) and structures which are inserted last can be largest (e.g. widest).

In an example, a middle embolic structure in an intrasacular stack of three embolic structures can be wider (e.g. 25% to 200% wider) than each of a proximal embolic structure and a distal embolic structure. This design can help to keep the structure in the aneurysm sac if the structure engages the walls of the aneurysm sac at its widest section (e.g. if the middle section of the sac is widest). This can be optimal for berry aneurysms. In an example, a proximal embolic structure can be wider (e.g. 25% to 200% wider) than each of a middle embolic structure and a distal embolic structure. This design can help to maximize coverage of the aneurysm neck to minimize flow of blood into the aneurysm sac. This design can also be optimal for fusiform aneurysms. In an example, proximal and distal embolic structures can each be wider (e.g. 25% to 200% wider) than a middle embolic structure. This design can help the device to bend laterally, which can be help to occlude asymmetric and/or irregularly-shaped aneurysms. In an example, the size of one or more embolic structures in a stack of embolic structures can be adjusted, controlled, and/or varied in real time by a device operator in order to best match the contours of a specific aneurysm sac.

In an example, embolic structures in a stack can all have the same level of flexibility, elasticity, and/or durometer. In an example, a middle embolic structure can have greater flexibility, greater elasticity, and/or lower durometer than each of a proximal embolic structure and a distal embolic structure. In an example, a distal embolic structure can have greater flexibility, greater elasticity, and/or lower durometer than each of a proximal embolic structure and a middle embolic structure. In an example, proximal and distal embolic structures can have greater flexibility, greater elasticity, and/or lower durometer than a middle embolic structure. In an example, embolic structures in a stack can all have the same porosity level. In an example, a middle embolic structure can have greater porosity than each of a proximal embolic structure and a distal embolic structure. In an example, a distal embolic structure can have greater porosity than each of a proximal embolic structure and a middle embolic structure.

In an example, some or all of the interiors of one or more embolic structures in a stack can be filled with embolic members and/or material after the one or more embolic structures have been inserted into an aneurysm sac. In an example, embolic members and/or material can be selected from the group consisting of: embolic coils, hydrogels, microsponges, beads, ribbons, string-of-pearls strands of embolic components, and congealing material. In an example, an embolic structure can further expand as the interior of the embolic member is filled with embolic material. This further expansion can help the embolic structure can better conform to the walls of an irregularly-shaped aneurysm sac and thus reduce the risk of recanalization.

In an example, there can be an opening (or hole) in an embolic structure. In an example, this opening can be centrally and/or axially located on the proximal surface of an embolic structure. In an example, this opening can be non-centrally and/or off-axially located on the proximal surface of an embolic structure. In an example, embolic members and/or material can be inserted through this opening into the interior of the embolic structure. In an example, embolic members and/or material can be inserted through this opening into the interiors of other, more-distal, embolic structures in a stack. In an example, all three embolic structures in a stack can be filled with embolic members and/or material. In an example, only one or two of three embolic structures in a stack may be filled with embolic members and/or material. In an example, there can be a tube, catheter, and/or channel through the center of a first embolic structure through which embolic material travels into the interior of more-distal second embolic structure, but not into the rest of the interior of the first embolic structure.

In an example, there can also be a valve which opens or closes the opening (or hole) in an embolic structure. In an example, this valve enables embolic members and/or material to enter, but not exit, the embolic structure. In an example, this valve can be controlled remotely by the device operator. In an example, a valve can be remotely opened and/or closed by an operator by the application of electromagnetic energy. In an example, a valve can be remotely opened and/or closed by an operator by pulling a filament. In an example, a valve can be remotely opened and/or closed by an operator by pushing, pulling, or rotating a wire. In an example, a valve can be remotely opened and/or closed by an operator by cutting, pulling, or pushing a flap or plug. In an example, a valve can passively open when embolic members and/or material is pushed through it and can passively close afterward. In an example, a valve can be a leaflet valve.

In an example, embolic structures in a stack of embolic structures can be pushed out of a catheter into an aneurysm sac by movement of a wire, inner catheter, or plunger. In an example, embolic structures in a stack of embolic structures can be moved out of a catheter into an aneurysm sac by microscale motor (e.g. a conveyer-belt MEMS). In an example, embolic structures in a stack of embolic structures can be moved out of a catheter into an aneurysm sac by a rotating helically-threaded mechanism (e.g. an Archimedes screw mechanism). In an example, embolic structures in a stack of embolic structures can be pushed out of a catheter by fluid pressure. In an example, embolic structures in a stack of embolic structures can be moved out a catheter in a flow of liquid (e.g. a saline solution). Example variations discussed elsewhere in this disclosure or priority-linked disclosures can also be applied to this example where relevant.

FIGS. 33 through 36 also show a method of creating and deploying an aneurysm occlusion device with a stack of embolic members comprising: radially-constraining a tubular mesh at multiple locations along its longitudinal axis, including both ends and at least one location between the ends, in order to create a stack of generally-spherical lobes; radially-compressing and longitudinally-elongating the stack for delivery through a catheter into a aneurysm sac; and allowing the stack to self-expand radially and shrink longitudinally in the aneurysm sac.

In an example, an intrasacular aneurysm occlusion device with a stack of embolic structures can comprise: a longitudinal catheter that is configured to be inserted into a blood vessel, wherein the catheter has a longitudinal axis spanning from its proximal end to its distal end and wherein the distal end is first inserted into the blood vessel; and a plurality of longitudinally-linked configuration-changing embolic structures which are configured to travel through the longitudinal catheter and to be inserted into an aneurysm; wherein each shape-changing embolic structure has its own internally-referenced Z axis, X axis, and Y axis; wherein its Z axis is substantially parallel to the longitudinal axis of the longitudinal catheter as the embolic structure travels through the longitudinal catheter, its X axis is substantially perpendicular to its Z axis, and its Y axis is substantially perpendicular to both its Z axis and X axis; wherein each configuration-changing embolic structure has a first configuration as the member travels through the longitudinal catheter and a second configuration within the aneurysm after it exits the longitudinal catheter; wherein the distance of the embolic structure spanning its Z axis is greater than the distance of the embolic structure spanning its X axis or Y axis in the first configuration; wherein the distance of the embolic structure spanning its Z axis is less than the distance of the embolic structure spanning its X axis or Y axis in the second configuration; wherein the cross-sectional shape of the embolic structure in an X-Z plane is substantially elliptical, oval, or another arcuate non-circular shape in the first configuration, with the longer dimension of the ellipse, oval, or another arcuate non-circular shape being along its Z axis; and wherein the cross-sectional shape of the embolic structure in the X-Z plane is substantially elliptical, oval, or another arcuate non-circulate shape in the second configuration, with the longer dimension of the ellipse, oval, or another arcuate non-circular shape being along its X axis.

In an example, an intrasacular aneurysm occlusion device with a stack of embolic structures can comprise: a proximal embolic structure which is configured to be deployed in an aneurysm sac at a first distance from the aneurysm neck; a middle embolic structure which is configured to be deployed in the aneurysm sac at a second distance from the aneurysm neck; and a distal embolic structure which is configured to be deployed in the aneurysm sac at a third distance from the aneurysm neck; wherein the second distance is greater than the first distance; wherein the third distance is greater than the second distance; and wherein the centers of the proximal embolic structure, the middle embolic structure, and the distal embolic structure are linearly-aligned.

In an example, the proximal embolic structure, the middle embolic structure, and the distal embolic structure can have ellipsoidal or oblate-spheroidal shapes. In an example, a first subset of the embolic structures can have ellipsoidal or oblate-spheroidal shapes and a second subset of the embolic structures can have toroidal or doughnut shapes. In an example, the proximal embolic structure, the middle embolic structure, and the distal embolic structure can have tree ornament shapes, wherein a tree ornament shape is defined as: an ellipsoid with an upward-facing spire and a downward-facing spire; or a 3D revolution of a single phase of a sine wave or normal curve. In an example, a first subset of the embolic structures can have ellipsoidal or oblate-spheroidal shapes and a second subset of the embolic structures can have tree-ornament shapes. In an example, the stack of embolic structures can be shaped like a 3D revolution of two or more phases of a sine wave around its central longitudinal axis.

In an example, the proximal embolic structure can be wider than the distal embolic structure. In an example, the middle embolic structure can be wider than the proximal embolic structure. In an example, there can be an opening in the proximal embolic structure through which embolic members and/or material is inserted into the interior of the proximal embolic structure. In an example, there can be an opening in the middle embolic structure through which embolic members and/or material is inserted into the interior of the middle embolic structure. In an example, there can be an opening in the distal embolic structure through which embolic members and/or material is inserted into the interior of the distal embolic structure. In an example, embolic members and/or material can be selected from the group consisting of: embolic coils, hydrogels, string-of-pearls embolic balls, and congealing material. In an example, there can be a valve in the opening. In an example, the valve can be remotely closed by the device operator. In an example, there can be a wire through the center of proximal embolic structure and the device operator can control the amount of radial expansion of one or more of the embolic structures by moving the wire.

In an example, an intrasacular aneurysm occlusion device with a stack of embolic structures can comprise: a catheter; a tubular mesh; and a plurality of bands, rings, and/or cylinders; wherein the tubular mesh is radially-constrained by the bands, rings, and/or cylinders at multiple locations along its longitudinal axis to create a stack of embolic structures; wherein the embolic structures are lobes, bulges, and/or segments of the tubular mesh; and wherein the stack of embolic structures is configured to be radially-compressed and longitudinally-extended for delivery through the catheter into an aneurysm sac and then radially-expanded and longitudinally-contracted within the aneurysm sac.

In an example, the embolic structures can be spherical before they are inserted into the catheter. In an example, the embolic structures can have ellipsoidal or oblate-spheroidal shapes after they have been inserted and expanded with the aneurysm sac. In an example, the embolic structures can have tree-ornament shapes, wherein a tree ornament shape is defined as: an ellipsoid with an upward-facing spire and a downward-facing spire; or a 3D revolution of a single phase of a sine wave or normal curve.

I claim:

1. An intrasacular aneurysm occlusion device with a stack of embolic structures comprising:
a longitudinal catheter that is configured to be inserted into a blood vessel, wherein the catheter has a longitudinal axis spanning from its proximal end to its distal end and wherein the distal end is first inserted into the blood vessel; and
a plurality of longitudinally-linked configuration-changing embolic structures which are configured to travel through the longitudinal catheter and to be inserted into an aneurysm;
wherein each shape-changing embolic structure has its own internally-referenced Z axis, X axis, and Y axis; wherein its Z axis is substantially parallel to the longitudinal axis of the longitudinal catheter as the embolic structure travels through the longitudinal catheter, its X axis is substantially perpendicular to its Z axis, and its Y axis is substantially perpendicular to both its Z axis and X axis;
wherein each configuration-changing embolic structure has a first configuration as the member travels through the longitudinal catheter and a second configuration within the aneurysm after it exits the longitudinal catheter;
wherein the distance of the embolic structure spanning its Z axis is greater than the distance of the embolic structure spanning its X axis or Y axis in the first configuration;
wherein the distance of the embolic structure spanning its Z axis is less than the distance of the embolic structure spanning its X axis or Y axis in the second configuration;
wherein the cross-sectional shape of the embolic structure in an X-Z plane is substantially elliptical, oval, or another arcuate non-circular shape in the first configuration, with the longer dimension of the ellipse, oval, or another arcuate non-circular shape being along its Z axis; and
wherein the cross-sectional shape of the embolic structure in the X-Z plane is substantially elliptical, oval, or another arcuate non-circulate shape in the second configuration, with the longer dimension of the ellipse, oval, or another arcuate non-circular shape being along its X axis.

2. An intrasacular aneurysm occlusion device with a stack of embolic structures comprising:
a proximal embolic structure which is configured to be deployed in an aneurysm sac at a first distance from the aneurysm neck;
a middle embolic structure which is configured to be deployed in the aneurysm sac at a second distance from the aneurysm neck; and
a distal embolic structure which is configured to be deployed in the aneurysm sac at a third distance from the aneurysm neck;
wherein the second distance is greater than the first distance;
wherein the third distance is greater than the second distance; and
wherein the centers of the proximal embolic structure, the middle embolic structure, and the distal embolic structure are linearly-aligned.

3. The device in claim 2 wherein the proximal embolic structure, the middle embolic structure, and the distal embolic structure have ellipsoidal or oblate-spheroidal shapes.

4. The device in claim 2 wherein a first subset of the embolic structures have ellipsoidal or oblate-spheroidal shapes and a second subset of the embolic structures have toroidal or doughnut shapes.

5. The device in claim 2 wherein the proximal embolic structure, the middle embolic structure, and the distal embolic structure have tree ornament shapes, wherein a tree ornament shape is defined as: an ellipsoid with an upward-facing spire and a downward-facing spire; or a 3D revolution of a single phase of a sine wave or normal curve.

6. The device in claim 2 wherein a first subset of the embolic structures have ellipsoidal or oblate-spheroidal shapes and a second subset of the embolic structures have tree-ornament shapes, wherein a tree ornament shape is defined as: an ellipsoid with an upward-facing spire and a downward-facing spire; or a 3D revolution of a single phase of a sine wave or normal curve.

7. The device in claim 2 wherein the stack of embolic structures is shaped like a 3D revolution of two or more phases of a sine wave around its central longitudinal axis.

8. The device in claim 2 wherein the proximal embolic structure is wider than the distal embolic structure.

9. The device in claim 2 wherein the middle embolic structure is wider than the proximal embolic structure.

10. The device in claim 2 wherein there is an opening in the proximal embolic structure through which embolic members and/or material is inserted into the interior of the proximal embolic structure.

11. The device in claim 10 wherein embolic members and/or material is selected from the group consisting of: embolic coils, hydrogels, string-of-pearls embolic balls, and congealing material.

12. The device in claim 2 wherein there is an opening in the middle embolic structure through which embolic members and/or material is inserted into the interior of the middle embolic structure.

13. The device in claim 2 wherein there is an opening in the distal embolic structure through which embolic members and/or material is inserted into the interior of the distal embolic structure.

14. The device in claim 10 wherein there is a valve in the opening.

15. The device in claim 14 wherein the valve can be remotely closed by the device operator.

16. The device in claim 2 wherein there is a wire through the center of proximal embolic structure and the device operator can control the amount of radial expansion of one or more of the embolic structures by moving the wire.

17. An intrasacular aneurysm occlusion device with a stack of embolic structures comprising:
a catheter;
a tubular mesh; and
a plurality of bands, rings, and/or cylinders;
wherein the tubular mesh is radially-constrained by the bands, rings, and/or cylinders at multiple locations along its longitudinal axis to create a stack of embolic structures;
wherein the embolic structures are lobes, bulges, and/or segments of the tubular mesh; and
wherein the stack of embolic structures is configured to be radially-compressed and longitudinally-extended for delivery through the catheter into an aneurysm sac and then radially-expanded and longitudinally-contracted within the aneurysm sac.

18. The device in claim 17 wherein the embolic structures are spherical before they are inserted into the catheter.

19. The device in claim 17 wherein the embolic structures have ellipsoidal or oblate-spheroidal shapes after they have been inserted and expanded with the aneurysm sac.

20. The device in claim 17 wherein the embolic structures have tree-ornament shapes, wherein a tree ornament shape is defined as: an ellipsoid with an upward-facing spire and a downward-facing spire; or a 3D revolution of a single phase of a sine wave or normal curve.
